(19) United States
(12) Patent Application Publication
(10) Pub. No.: US 2022/0370548 A1
(43) Pub. Date: Nov. 24, 2022

(54) NEUROLOGICAL DISEASE TREATMENT WITH COMPLEMENT INHIBITORS

(71) Applicant: RA PHARMACEUTICALS, INC., Cambridge, MA (US)

(72) Inventors: Simon J. READ, Lexington, MA (US); Alonso RICARDO, Winchester, MA (US); Camil SAYEGH, Belmont, MA (US); Xiangyang XU, Auburndale, MA (US); Yalan TANG, Cambridge, MA (US); Guo-Qing TANG, Acton, MA (US)

(21) Appl. No.: 17/642,096

(22) PCT Filed: Sep. 11, 2020
(86) PCT No.: PCT/US2020/050422

§ 371 Date: Mar. 10, 2022

Related U.S. Application Data
(60) Provisional application No. 62/899,488, filed on Sep. 12, 2019.
(60) Provisional application No. 62/969,742, filed on Feb. 4, 2020.
(60) Provisional application No. 62/988,587, filed on Mar. 12, 2020.
(60) Provisional application No. 63/021,742, filed on May 8, 2020.

(51) Int. Cl. A61K 38/12; A61P 21/00; G01N 33/68

(52) U.S. Cl. CPC A61K 38/12; A61P 21/00; G01N 33/6896; G01N 2800/2835; G01N 2800/52

(57) ABSTRACT

The present disclosure provides methods of treating inflammatory indications with complement inhibitor compounds and compositions. Included are compounds and methods for treating amyotrophic lateral sclerosis (ALS).

### CROSS REFERENCE TO RELATED APPLICATIONS

This application claims priority to U.S. Provisional Application No. 62/899,488 filed on Sep. 12, 2019 entitled NEUROLOGICAL DISEASE TREATMENT WITH COMPLEMENT INHIBITORS, U.S. Provisional Application No. 62/969,742 filed on Feb. 4, 2020 entitled NEUROLOGICAL DISEASE TREATMENT WITH COMPLEMENT INHIBITORS, U.S. Provisional Application No. 62/988,587 filed on Mar. 12, 2020 entitled NEUROLOGICAL DISEASE TREATMENT WITH COMPLEMENT INHIBITORS, and U.S. Provisional Application No. 63/021,742 filed on May 8, 2020 entitled NEUROLOGICAL DISEASE TREATMENT WITH COMPLEMENT INHIBITORS, the contents of each of which are herein incorporated by reference in their entirety.

### SEQUENCE LISTING

The present application is being filed along with a Sequence Listing in electronic format. The Sequence Listing file, entitled 2011_1058PCT_SL.txt, was created on Sep. 11, 2020 and is 1,231 bytes in size. The information in electronic format of the Sequence Listing is incorporated herein by reference in its entirety.

### BACKGROUND

The vertebrate immune response is comprised of adaptive and innate immune components. While the adaptive immune response is selective for particular pathogens and is slow to respond, components of the innate immune response recognize a broad range of pathogens and respond rapidly upon infection. One such component of the innate immune response is the complement system.

The complement system includes about 20 circulating complement component proteins, synthesized primarily by the liver. Components of this particular immune response were first termed “complement” due to the observation that they complemented the antibody response in the destruction of bacteria. These proteins remain in an inactive form prior to activation in response to infection. Activation occurs by way of a pathway of proteolytic cleavage initiated by pathogen recognition and leading to pathogen destruction. Three such pathways are known in the complement system and are referred to as the classical pathway, the lectin pathway, and the alternative pathway. The classical pathway is activated when an IgG or IgM molecule binds to the surface of a pathogen. The lectin pathway is initiated by the mannan-binding lectin protein recognizing the sugar residues of a bacterial cell wall. The alternative pathway remains active at low levels in the absence of any specific stimuli. While all three pathways differ with regard to initiating events, all three pathways converge with the cleavage of complement component C3. C3 is cleaved into two products termed C3a and C3b. Of these, C3b becomes covalently linked to the pathogen surface while C3a acts as a diffusible signal to promote inflammation and recruit circulating immune cells. Surface-associated C3b forms a complex with other components to initiate a cascade of reactions among the later components of the complement system. Due to the requirement for surface attachment, complement activity remains localized and minimizes destruction to non-target cells.

Pathogen-associated C3b facilitates pathogen destruction in two ways. In one pathway, C3b is recognized directly by phagocytic cells and leads to engulfment of the pathogen. In the second pathway, pathogen-associated C3b initiates the formation of the membrane attack complex (MAC). In the first step, C3b complexes with other complement components to form the C5-convertase complex. Depending on the initial complement activation pathway, the components of this complex may differ. C5-convertase formed as the result of the classical complement pathway comprises C4b and C2a in addition to C3b. When formed by the alternative pathway, C5-convertase comprises two subunits of C3b as well as one Bb component.

Complement component C5 is cleaved by either C5-convertase complex into C5a and C5b. C5a, much like C3a, diffuses into the circulation and promotes inflammation, acting as a chemoattractant for inflammatory cells. C5b remains attached to the cell surface where it triggers the formation of the MAC through interactions with C6, C7, C8 and C9. The MAC is a hydrophilic pore that spans the membrane and promotes the free flow of fluid into and out of the cell, thereby destroying it.

An important component of all immune activity is the ability of the immune system to distinguish between self and non-self cells. Pathology arises when the immune system is unable to make this distinction. In the case of the complement system, vertebrate cells express proteins that protect them from the effects of the complement cascade. This ensures that targets of the complement system are limited to pathogenic cells. Many complement-related disorders and diseases are associated with inflammation that leads to or arises from abnormal destruction of self cells by the complement cascade.

Amyotrophic lateral sclerosis (ALS) is a progressive disease causing bodily muscle weakness. Disease progression varies between individuals with three-year median survival with onset of symptoms and 80% patient death within five years. Although a specific cause of disease is unknown, complement-related immune dysregulation is thought to be a contributing factor. Complement components are synthesized by cells of the central nervous system, including neurons, astrocytes, oligodendrocytes, and microglia. Further, biomarkers of classical complement activation pathway (C1q and C4) have been identified in association ALS serum, cerebrospinal fluid, motor cortex and spinal cord parenchyma, and skeletal muscle tissue.

There remains a need in the field for compounds and therapeutic methods for addressing complement-related diseases and disorders, including those affecting the nervous system, such as ALS. The present disclosure meets this need by providing related compounds and methods of treatment.

### SUMMARY

In some embodiments, the present disclosure provides a method of treating a subject, wherein the subject has or is suspected of having amyotrophic lateral sclerosis (ALS) by administering a complement inhibitor to the subject, wherein the complement inhibitor reduces, prevents, stabilizes, or reverses one or more effects of ALS experienced by the subject. The complement inhibitor may be a C5 inhibitor. The complement inhibitor may be zilucoplan. Prior to treatment, subject ALS disease severity may be elevated and/or may be increasing at a specific rate. Complement inhibitor treatment may stabilize or reduce subject ALS disease severity and/or reduce the specific rate at which subject ALS disease severity is increasing. Change in subject ALS disease severity may be measured by ALS Functional Rating Scale-Revised (ALSFRS-R). Prior to treatment, subject respiratory function may be decreased and/or declining at a specific rate. Complement inhibitor treatment may stabilize or improve subject respiratory function and/or reduce the specific rate at which subject respiratory function is declining. Change in subject respiratory function may be assessed by slow vital capacity (SVC). Prior to treatment, subject muscle strength may be decreased and/or declining at a specific rate. Complement inhibitor treatment may stabilize or improve subject muscle strength and/or reduce the specific rate at which subject muscle strength is declining. Change in subject muscle strength may be measured by hand held dynamometry (HHD). Complement inhibitor treatment may stabilize or improve subject speech capability and/or stabilize or improve a rate of subject speech capability decline. Subject speech capability may include one or more of articulatory precision, speaking rate, percent of speech signal voiced, phonation length, consonant length, and nasality. Complement inhibitor treatment may stabilize or improve subject ALS biomarker profile and/or rate of subject ALS biomarker profile change. The subject ALS biomarker profile may include a biomarker selected from one or more of neurofilament heavy chain (pNfH), neurofilament light chain (NfL), C5, and a complement activity biomarker. The complement activity biomarker may be selected from one or more of C5a, C5b-9, soluble C5b-9, C3a, C4a, Ba, Bb, Factor I, and Factor H. The complement activity biomarker may be detected in subject cerebrospinal fluid (CSF), plasma, and/or serum. The subject may have or may be suspected of having myasthenia gravis. The subject may be positive for at least one autoantibody. The at least one autoantibody may be one or more of anti-acetylcholine receptor antibody, anti-LDL receptor related protein 4, anti-agrin, and anti-muscle associated receptor tyrosine kinase. The subject may have impaired blood brain barrier function. The impaired blood brain barrier function may be assessed by determining the subject's albumin quotient. The subject may be determined to have definite ALS or probable ALS based on revised El Escorial criteria.

In some embodiments, the present disclosure provides a method of diagnosing ALS in a subject by detecting and/or quantifying complement activity associated with the subject or a biological sample from the subject and diagnosing the subject with ALS based on complement activity detected and/or quantified. Complement activity detection and/or quantification may include detecting and/or quantifying a complement activity biomarker. The complement activity biomarker may be selected from one or more of C5a, C5b-9, soluble C5b-9, C3a, C4a, Ba, Bb, Factor I, and Factor H. The complement activity biomarker may be detected and/or quantified in subject CSF, plasma, and/or serum.

In some embodiments, the present disclosure provides a method of monitoring ALS in a subject, the method including (1) detecting and/or quantifying complement activity associated with: (a) the subject at an initial time point or over an initial time period; or (b) a biological sample obtained from the subject at the initial time point or over the initial time period; and (2) detecting and/or quantifying complement activity associated with: (a) the subject at a subsequent time point or over a subsequent time period; or (b) a biological sample obtained from the subject at the subsequent time point or over the subsequent time period; and (3) monitoring ALS in the subject by assessing any change in complement activity detected and/or quantified between the initial time point or period and the subsequent time point or period. Complement activity detection and/or quantification may include detecting and/or quantifying a complement activity biomarker. The complement activity biomarker may be selected from one or more of C5a, C5b-9, soluble C5b-9, C3a, C4a, Ba, Bb, Factor I, and Factor H. The complement activity biomarker may be detected and/or quantified in subject CSF, plasma, and/or serum. The subject may be determined to have definite ALS or probable ALS based on revised El Escorial criteria.

In some embodiments, the present disclosure provides a method of stratifying subjects with ALS or suspected of having ALS into two or more groups, the method comprising detecting and/or quantifying complement activity associated with each subject or a biological sample from each subject and assigning each subject to at least one of the two or more groups based on complement activity detected and/or quantified. Complement activity detection and/or quantification may include detecting and/or quantifying a complement activity biomarker. The complement activity biomarker may be selected from one or more of C5a, C5b-9, soluble C5b-9, C3a, C4a, Ba, Bb, Factor I, and Factor H. The complement activity biomarker may be detected and/or quantified in subject CSF, plasma, and/or serum. Subjects may be assigned to a treatment group based on CSF, plasma, and/or serum complement activity biomarker levels that are elevated in comparison to CSF, plasma, and/or serum complement activity biomarker levels in subjects without ALS. The treatment group may be characterized by eligibility for complement inhibitor treatment. The complement inhibitor treatment may include zilucoplan treatment. The subject may be determined to have definite ALS or probable ALS based on revised El Escorial criteria.

The dose and/or regimen of complement inhibitor administered to a subject according to treatment methods described herein may be determined based on the level of a complement activity biomarker detected in CSF, plasma, and/or serum obtained from the subject prior to treatment. The complement activity biomarker detected in CSF, plasma, and/or serum obtained from the subject prior to treatment may be selected from one or more of C5a, C5b-9, soluble C5b-9, C3a, C4a, Ba, Bb, Factor I, and Factor H. The dose and/or regimen of complement inhibitor administered may be adjusted after one or more prior complement inhibitor administration based on the level of a complement activity biomarker detected in CSF, plasma, and/or serum obtained from the subject after one or more prior administration. The complement activity biomarker detected in CSF, plasma, and/or serum obtained from the subject after one or more prior administration may be selected from one or more of C5a, C5b-9, soluble C5b-9, C3a, C4a, Ba, Bb, Factor I, and Factor H. The complement inhibitor administered may be zilucoplan.

In some embodiments, the present disclosure provides a method of treating a subject having a complement-related indication, wherein the subject has or is suspected of having an impaired blood brain barrier. The method may include administering a complement inhibitor to the subject. The complement inhibitor may reduce, prevent, stabilize, or reverse one or more effects of the complement-related indication. The complement inhibitor may include a C5 inhibitor. The complement inhibitor may include zilucoplan. The complement-related indication may include a neuromuscular inflammatory indication. The neuromuscular inflammatory indication may include ALS. The subject may be determined to have definite ALS or probable ALS based on revised El Escorial criteria. The subject may be assessed for blood brain barrier impairment by determining the subject's albumin quotient.

In some embodiments, the present disclosure provides a method of selecting a subject for treatment with a complement inhibitor, wherein the subject has or is suspected of having ALS, the method including assessing the presence or level of at least one biomarker in a subject sample and selecting the subject for treatment with the complement inhibitor based on the assessment of the presence or level of the at least one biomarker in the subject sample. The at least one biomarker may include one or more of a neuroaxonal degeneration biomarker, a neuroinflammation biomarker, and a complement activity biomarker. The subject sample may include a plasma sample and/or a CSF sample. The neuroaxonal degeneration biomarker may be pNfH and/or NfL. The neuroinflammation biomarker may include monocyte chemoattractant protein-1 (MCP-1), chitotriosidase-1 (CHIT1), and/or chitinase-3-like protein 1 (YKL-40). The complement activity biomarker may include C5a, C5b-9, sC5b-9, C5b6 complex, C3a, C4a, Ba, Bb, Factor I, and/or Factor H. The complement inhibitor may include zilucoplan. Levels of one or more of Nfl, pNfH, CHIT1, C5a, and sC5b-9 may be elevated in the subject sample relative to levels in a subject sample from a subject without ALS. The subject sample and the subject sample from a subject without ALS may include CSF. The subject sample may be obtained from a subject with impaired blood brain barrier function. One or more of C3a, Bb, and Ba may be elevated in the subject sample relative to levels in a subject sample from a subject without ALS. The subject sample and the subject sample from a subject without ALS may include plasma. C3a levels may be elevated in the subject sample relative to levels in a subject sample from a subject without ALS.

In some embodiments, the present disclosure provides a method of treating a subject with ALS or suspected of having ALS, the method including selecting the subject for treatment with a complement inhibitor according to any of the methods described herein [e.g., based on assessing the presence or level of at least one biomarker (e.g., a neuroaxonal degeneration biomarker, a neuroinflammation biomarker, and/or a complement activity biomarker) and administering the complement inhibitor to the subject. The complement inhibitor may include zilucoplan.

In some embodiments, the present disclosure provides a composition comprising a complement inhibitor for use in a method of treating a subject having or suspected of having ALS, wherein the composition reduces, prevents, stabilizes, or reverses one or more effects of ALS experienced by the subject. The complement inhibitor may include a C5 inhibitor. The complement inhibitor may include zilucoplan. Prior to treatment, subject ALS disease severity may be elevated and/or may be increasing at a specific rate. The treatment may stabilize or reduce subject ALS disease severity and/or reduce the specific rate at which subject ALS disease severity is increasing. Change in subject ALS disease severity may be measured by ALSFRS-R. Prior to treatment, subject respiratory function may be decreased and/or may be declining at a specific rate. The treatment with the complement inhibitor may stabilize or improve subject respiratory function and/or reduce the specific rate at which subject respiratory function is declining. Change in subject respiratory function may be assessed by SVC. Prior to treatment, subject muscle strength may be decreased and/or declining at a specific rate and treatment with the composition may stabilize or improve subject muscle strength and/or reduce the specific rate at which subject muscle strength is declining. Change in subject muscle strength may be measured by HHD. The treatment may stabilize or improve subject speech capability and/or rate of subject speech capability decline. Subject speech capability may include one or more of articulatory precision, speaking rate, percent of speech signal voiced, phonation length, consonant length, and nasality. The treatment may stabilize or improve subject ALS biomarker profile and/or rate of subject ALS biomarker profile change. The subject ALS biomarker profile may include a biomarker selected from one or more of pNfH, NfL, C5, and a complement activity biomarker. The complement activity biomarker may be selected from one or more of C5a, C5b-9, soluble C5b-9, C3a, C4a, Ba, Bb, Factor I, and Factor H. The complement activity biomarker may be detected in subject CSF, plasma, and/or serum. The dose and/or regimen of the composition may be adjusted based on the level of a complement activity biomarker detected in a CSF sample, plasma sample, and/or serum sample obtained from the subject after one or more prior composition administration. The complement activity biomarker detected in the CSF sample, plasma sample, and/or serum obtained from the subject after one or more prior composition administration may be selected from one or more of C5a, C5b-9, soluble C5b-9, C3a, C4a, Ba, Bb, Factor I, and Factor H. The subject may have or be suspected of having myasthenia gravis. The subject may be positive for at least one autoantibody. The at least one autoantibody may include one or more of anti-acetylcholine receptor antibody, anti-LDL receptor related protein 4, anti-agrin, and anti-muscle associated receptor tyrosine kinase. The subject may have impaired blood brain barrier function. The impaired blood brain barrier function may be assessed by determining the subject's albumin quotient. The subject may be determined to have definite ALS or probable ALS based on revised El Escorial criteria.

In some embodiments, the present disclosure provides a method of diagnosing ALS in a subject by detecting and/or quantifying complement activity associated with a biological sample obtained from the subject and diagnosing the subject with ALS based on complement activity detected and/or quantified. The step of detecting and/or quantifying complement activity associated with the biological sample may include detecting and/or quantifying a complement activity biomarker in the biological sample. The complement activity biomarker may be selected from one or more of C5a, C5b-9, soluble C5b-9, C3a, C4a, Ba, Bb, Factor I, and Factor H. The biological sample may include subject CSF, plasma, and/or serum.

In some embodiments, the present disclosure provides a method of monitoring ALS in a subject by detecting and/or quantifying complement activity associated with a biological sample obtained from the subject at an initial time point or over an initial time period; detecting and/or quantifying complement activity associated with a biological sample obtained from the subject at a subsequent time point or over a subsequent time period; and monitoring ALS in the subject by assessing any change in complement activity detected and/or quantified between the biological sample obtained from the subject at the initial time point or over the initial time period and the biological sample obtained from the subject at the subsequent time point or over the subsequent time period. Complement activity detection and/or quantification may include detecting and/or quantifying a complement activity biomarker. The complement activity biomarker may be selected from one or more of C5a, C5b-9, soluble C5b-9, C3a, C4a, Ba, Bb, Factor I, and Factor H. The biological sample obtained from the subject at the initial time point or over the initial time period and/or the biological sample obtained from the subject at the subsequent time point or over the subsequent time period may include CSF, plasma, and/or serum. The subject may have definite ALS or probable ALS, as assessed based on revised El Escorial criteria.

In some embodiments, the present disclosure provides a method of stratifying subjects with ALS or suspected of having ALS into two or more groups by detecting and/or quantifying complement activity associated with a biological sample obtained from each subject and assigning each subject to at least one of the two or more groups based on complement activity detected and/or quantified. Complement activity detection and/or quantification may include detecting and/or quantifying a complement activity biomarker. The complement activity biomarker may be selected from one or more of C5a, C5b-9, soluble C5b-9, C3a, C4a, Ba, Bb, Factor I, and Factor H. The biological sample obtained from each subject may include CSF, plasma, and/or serum. The two or more groups may include a treatment group, wherein the subjects are assigned to the treatment group based on CSF, plasma, and/or serum complement activity biomarker levels that are elevated in comparison to CSF, plasma, and/or serum complement activity biomarker levels in subjects without ALS. The treatment group may be characterized by eligibility for complement inhibitor treatment. The complement inhibitor treatment may include zilucoplan treatment. The subject may be determined to have definite ALS or probable ALS based on revised El Escorial criteria. The dose and/or regimen of the composition may be determined based on the level of a complement activity biomarker detected in a CSF sample, plasma sample, and/or serum obtained from the subject prior to treatment. The complement activity biomarker detected in the CSF sample, plasma sample, and/or serum obtained from the subject prior to treatment may be selected from one or more of C5a, C5b-9, soluble C5b-9, C3a, C4a, Ba, Bb, Factor I, and Factor H.

In some embodiments, the present disclosure provides a composition that includes a complement inhibitor for use in a method of treating a subject having a complement-related indication, wherein the subject has or is suspected of having an impaired blood brain barrier, wherein the composition reduces, prevents, stabilizes, or reverses one or more effects of the complement-related indication. The complement inhibitor may be a C5 inhibitor. The complement inhibitor may be zilucoplan. The complement-related indication may be a neuromuscular inflammatory indication. The neuromuscular inflammatory indication may be ALS. The subject may be determined to have definite ALS or probable ALS based on revised El Escorial criteria. The subject may be assessed for blood brain barrier impairment by determining the subject's albumin quotient.

In some embodiments, the present disclosure provides a composition that includes a complement inhibitor for use in a method of treating a subject with ALS or suspected of having ALS, the method including assessing the presence or level of at least one biomarker in a subject sample and selecting the subject for treatment with the complement inhibitor based on the assessment of the presence or level of the at least one biomarker in the subject sample. The at least one biomarker may include one or more of a neuroaxonal degeneration biomarker, a neuroinflammation biomarker, and a complement activity biomarker. The subject sample may include a plasma sample and/or a CSF sample. The neuroaxonal degeneration biomarker may be pNfH and/or NfL. The neuroinflammation biomarker may include MCP-1, CHIT1, and/or YKL-40. The complement activity biomarker may include C5a, C5b-9, sC5b-9, C5b6 complex, C3a, C4a, Ba, Bb, Factor I, and/or Factor H. Levels of one or more of Nfl, pNfH, CHIT1, C5a, and sC5b-9 may be elevated in the subject sample (e.g., CSF) relative to levels in a subject sample from a subject without ALS. The subject sample may be obtained from a subject with impaired blood brain barrier function. One or more of C3a, Bb, and Ba may be elevated in the subject sample (e.g., plasma) relative to levels in a subject sample from a subject without ALS. C3a levels may be elevated in the subject sample relative to levels in a subject sample from a subject without ALS. The complement inhibitor may include a C5 inhibitor. The C5 inhibitor may include zilucoplan.

### BRIEF FIGURE DESCRIPTION

The foregoing and other objects, features and advantages of particular embodiments of the disclosure will be apparent from the following description and illustrations in the accompanying figures.

FIG. 1 is a graph showing fold change in CSF biomarker levels from subjects with impaired blood brain barrier (as determined by CSF albumin:plasma albumin ratio). Statistical analysis was performed using Kruskal-Wallis test (*p<0.05, **p<0.01, ***p<0.001, ****p<0.0001). Whiskers indicate 5-95 percentile and “+” within boxes denotes mean.

FIG. 2 is a graph showing fold change in CSF biomarker levels from subjects with intact blood brain barrier (as determined by CSF albumin:plasma albumin ratio). Statistical analysis was performed using Kruskal-Wallis test (*p<0.05, **p<0.01, ***p<0.001, ****p<0.0001). Whiskers indicate 5-95 percentile and “+” within boxes denotes mean.

FIG. 3 is a graph showing median levels of C5a in plasma samples from healthy control subjects compared with median levels in subjects with definite or probable ALS. Error bars indicate 95% confidence interval. Significance value was determined by Mann-Whitney test with p>0.05 threshold.

FIG. 4 is a graph showing median levels of sC5b9 in plasma samples from healthy control subjects compared with median levels in subjects with definite or probable ALS. Error bars indicate 95% confidence interval. Significance value was determined by Mann-Whitney test with p>0.05 threshold.

FIG. 5 is a graph showing median levels of C3a in plasma samples from healthy control subjects compared with median levels in subjects with definite or probable ALS. Error bars indicate 95% confidence interval. Significance value was determined by Mann-Whitney test with p>0.05 threshold.

FIG. 6 is a graph showing median levels of C4a in plasma samples from healthy control subjects compared with median levels in subjects with definite or probable ALS. Error bars indicate 95% confidence interval. Significance value was determined by Mann-Whitney test with p>0.05 threshold.

FIG. 7 is a graph showing median levels of Ba in plasma samples from healthy control subjects compared with median levels in subjects with definite or probable ALS. Error bars indicate 95% confidence interval. Significance value was determined by Mann-Whitney test with p>0.05 threshold.

FIG. 8 is a graph showing median levels of Bb in plasma samples from healthy control subjects compared with median levels in subjects with definite or probable ALS. Error bars indicate 95% confidence interval. Significance value was determined by Mann-Whitney test with p>0.05 threshold.

FIG. 9 is a graph showing median levels of fH in plasma samples from healthy control subjects compared with median levels in subjects with definite or probable ALS. Error bars indicate 95% confidence interval. Significance value was determined by Mann-Whitney test with p>0.05 threshold.

FIG. 10 is a graph showing median levels of fI in plasma samples from healthy control subjects compared with median levels in subjects with definite or probable ALS. Error bars indicate 95% confidence interval. Significance value was determined by Mann-Whitney test with p>0.05 threshold.

FIG. 11 is a graph showing median levels of C5a in CSF samples from healthy control subjects compared with median levels in subjects with definite or probable ALS. Error bars indicate 95% confidence interval. Significance value was determined by Mann-Whitney test with p>0.05 threshold.

FIG. 12 is a graph showing median levels of sC5b9 in CSF samples from healthy control subjects compared with median levels in subjects with definite or probable ALS. Error bars indicate 95% confidence interval. Significance value was determined by Mann-Whitney test with p>0.05 threshold.

FIG. 13 is a graph showing median levels of C3a in CSF samples from healthy control subjects compared with median levels in subjects with definite or probable ALS. Error bars indicate 95% confidence interval. Significance value was determined by Mann-Whitney test with p>0.05 threshold.

FIG. 14 is a graph showing median levels of C4a in CSF samples from healthy control subjects compared with median levels in subjects with definite or probable ALS. Error bars indicate 95% confidence interval. Significance value was determined by Mann-Whitney test with p>0.05 threshold.

FIG. 15 is a graph showing median levels of Ba in CSF samples from healthy control subjects compared with median levels in subjects with definite or probable ALS. Error bars indicate 95% confidence interval. Significance value was determined by Mann-Whitney test with p>0.05 threshold.

FIG. 16 is a graph showing median levels of Bb in CSF samples from healthy control subjects compared with median levels in subjects with definite or probable ALS. Error bars indicate 95% confidence interval. Significance value was determined by Mann-Whitney test with p>0.05 threshold.

FIG. 17 is a graph showing median levels of fH in CSF samples from healthy control subjects compared with median levels in subjects with definite or probable ALS. Error bars indicate 95% confidence interval. Significance value was determined by Mann-Whitney test with p>0.05 threshold.

FIG. 18 is a graph showing median levels of fI in CSF samples from healthy control subjects compared with median levels in subjects with definite or probable ALS. Error bars indicate 95% confidence interval. Significance value was determined by Mann-Whitney test with p>0.05 threshold.

FIG. 19 is a graph showing albumin quotient values determined using plasma and CSF samples from healthy control subjects and subjects with definite or probable ALS. Levels associated with intact, mildly impaired, or moderately impaired blood brain barrier are indicated. Error bars indicate 95% confidence interval. Significance value was determined by Mann-Whitney test with p>0.05 threshold.

FIG. 20 is a graph showing neurofilament light chain (NfL) and phosphorylated neurofilament heavy chain (pNfH) biomarker levels in CSF from healthy control subjects and subjects with definite or probable ALS. For NfL in ALS, N=28 and for pNfH in ALS, N=17. Error bars indicate 95% confidence interval. Significance values were determined by Mann-Whitney test (2 groups) or Kruskal-Wallis test (3 groups).

FIG. 21 is a graph showing chitotriosidase-1 (CHIT1) and chitinase-3-like protein 1 (YKL-40) biomarker levels in CSF from healthy control subjects and subjects with ALS (N=17). Error bars indicate 95% confidence interval. Significance values were determined by Mann-Whitney test (2 groups) or Kruskal-Wallis test (3 groups). “ns” indicates no significant difference.

### DETAILED DESCRIPTION

### Introduction

Embodiments of the present disclosure relate to compounds and compositions for modulating complement activity and related methods of use. Complement activity protects the body from foreign pathogens but can lead to self-cell destruction with elevated activity or poor regulation. Complement modulators may be complement inhibitors, such as zilucoplan. Zilucoplan is a synthetic, macrocyclic peptide that binds complement component 5 (C5) with sub-nanomolar affinity and allosterically inhibits its cleavage into C5a and C5b upon activation of the classical, alternative, or lectin pathways (see, e.g., U.S. Pat. No. 10,106,579, the contents of which are herein incorporated by reference in their entirety).

Included herein are methods of treating complement-related indications by administering complement inhibitors (e.g., zilucoplan). Such complement-related indications include neuromuscular inflammatory indications, including amyotrophic lateral sclerosis (ALS). Complement inhibitors may be used to treat these conditions by reducing or eliminating the harmful effects of complement-mediated self-tissue destruction associated with overactive complement. These and other embodiments of the disclosure are described in detail below.

### I. Compounds and Compositions

In some embodiments, the present disclosure provides compounds and compositions which function to modulate complement activity. Such compounds and compositions may include inhibitors that block complement activation. As used herein, “complement activity” includes the activation of the complement cascade, the formation of cleavage products from a complement component such as C3 or C5, the assembly of downstream complexes following a cleavage event, or any process or event attendant to, or resulting from, the cleavage of a complement component, e.g., C3 or C5. Complement inhibitors may include C5 inhibitors that block complement activation at the level of complement component C5. C5 inhibitors may bind C5 and prevent its cleavage, by C5 convertase, into the cleavage products C5a and C5b. As used herein, “Complement component C5” or “C5” is defined as a complex which is cleaved by C5 convertase into at least the cleavage products, C5a and C5b. “C5 inhibitors,” as referred to herein, include any compound or composition that inhibits the processing or cleavage of the pre-cleaved complement component C5 complex or the cleavage products of the complement component C5.

It is understood that inhibition of C5 cleavage prevents the assembly and activity of the cytolytic membrane attack complex (MAC) on glycosylphosphatidylinositol (GPI) adherent protein-deficient erythrocytes. In some cases, C5 inhibitors presented herein may also bind C5b, preventing C6 binding and subsequent assembly of the C5b-9 MAC.

C5 inhibitor compounds may include, but are not limited to, any of those presented in Table 1. References listed and information supporting listed clinical study numbers are incorporated herein by reference in their entirety.

TABLE 1
C5 inhibitors

| | | | | Clinical study | |
|---|---|---|---|---|---|
| Compound | Company | Target | Compound type | numbers | References |
| Eculizumab | Alexion | C5 | Monoclonal antibody | NCT01303952; | U.S. Patent Nos. |
| (SOLIRIS ®) | Pharmaceuticals, | | directed against C5 | NCT02093533; | 6,355,245; |
| | Inc. | | protein. Inhibits C5 | NCT01567085; | 9,732,149; |
| | | | cleavage. | NCT01919346; | 9,718,880 |
| | | | | NCT01895127; | |
| | | | | NCT01399593; | |
| | | | | NCT02145182; | |
| | | | | NCT01106027; | |
| | | | | NCT02301624; | |
| | | | | NCT01997229; | |
| | | | | NCT01892345 | |
| ALXN1210 | Alexion | C5 | Antibody | NCT02598583; | U.S. |
| | Pharmaceuticals, | | | NCT02605993; | 2016/0168237 |
| | Inc. | | | NCT02946463; | |
| | | | | NCT03056040; | |
| | | | | NCT02949128 | |
| Tesidolumab/L | Novartis | C5 | Antibody | NCT02878616; | U.S. Pat. No. 8,241,628; |
| FG316 | | | | NCT02763644; | U.S. Pat. No. 8,883,158 |
| | | | | NCT01527500; | |
| | | | | NCT02515942; | |
| | | | | NCT02534909; | |
| | | | | NCT01526889 | |
| ALN-CC5 | Alnylam | C5 | Nucleic acid | NCT02352493 | |
| Zimura | Ophthotech | C5 | Nucleic acid | NCT02397954; | |
| | | | | NCT02686658 | |
| Coversin | Akari | C5 | Protein | NCT02591862 | |
| ALXN1007 | Alexion | C5a | Antibody | NCT02245412; | |
| | | | | NCT02128269 | |
| IFX-1 | InflaRx | C5a | Antibody | NCT02246595; | |
| | | | | NCT02866825; | |
| | | | | NCT03001622 | |
| MUBODINA ® | Adienne Pharma | C5 | Antibody | | U.S. Pat. No. 7,999,081 |
| ALXN5500 | Alexion | C5 | Antibody | | |
| | Pharmaceuticals, | | | | |
| | Inc. | | | | |
| ISU305 | ISU ABXIS | C5 | Antibody | | |
| Long-acting | Akari | C5 | Protein | | |
| coversin | | | | | |
| SOBI005 | Swedish Orphan | C5 | Protein | | |
| | Biovitrum Ab | | | | |
| IFX-2, IFX-3 | InflaRx | C5a | Antibody | | |
| NOX-D21 | Noxxon | C5a | Spiegelmer | | |
| rEV576 | Volution | C5 | Antibody | | Penabad et al., |
| | Immunopharmaceuticals | | | | Lupus, 2014 |
| | | | | | 23(12): 1324-6 |
| ARC1005 | Novo Nordisk | C5 | Antibody | | |
| SOMAmers | SomaLogic | C5 | Antibody | | |

### Peptide-Based Compounds

In some embodiments, C5 inhibitors of the present disclosure are polypeptides. According to the present disclosure, any amino acid-based molecule (natural or non-natural) may be termed a “polypeptide” and this term embraces “peptides,” “peptidomimetics,” and “proteins.” “Peptides” are traditionally considered to range in size from about 4 to about 50 amino acids. Polypeptides larger than about 50 amino acids are generally termed “proteins.”

C5 inhibitor polypeptides may be linear or cyclic. Cyclic polypeptides include any polypeptides that have as part of their structure one or more cyclic features such as a loop and/or an internal linkage. In some embodiments, cyclic polypeptides are formed when a molecule acts as a bridging moiety to link two or more regions of the polypeptide. As used herein, the term “bridging moiety” refers to one or more components of a bridge formed between two adjacent or non-adjacent amino acids, non-natural amino acids or non-amino acids in a polypeptide. Bridging moieties may be of any size or composition. In some embodiments, bridging moieties may include one or more chemical bonds between two adjacent or non-adjacent amino acids, non-natural amino acids, non-amino acid residues or combinations thereof. In some embodiments, such chemical bonds may be between one or more functional groups on adjacent or non-adjacent amino acids, non-natural amino acids, non-amino acid residues or combinations thereof. Bridging moieties may include one or more of an amide bond (lactam), disulfide bond, thioether bond, aromatic ring, triazole ring, and hydrocarbon chain. In some embodiments, bridging moieties include an amide bond between an amine functionality and a carboxylate functionality, each present in an amino acid, non-natural amino acid or non-amino acid residue side chain. In some embodiments, the amine or carboxylate functionalities are part of a non-amino acid residue or non-natural amino acid residue.

C5 inhibitor polypeptides may be cyclized through the carboxy terminus, the amino terminus, or through any other convenient point of attachment, such as, for example, through the sulfur of a cysteine (e.g., through the formation of disulfide bonds between two cysteine residues in a sequence) or any side-chain of an amino acid residue. Further linkages forming cyclic loops may include, but are not limited to, maleimide linkages, amide linkages, ester linkages, ether linkages, thiol ether linkages, hydrazone linkages, or acetamide linkages.

In some embodiments, peptides may be synthesized on solid supports (e.g., rink amide resin) via solid phase peptide synthesis (SPPS). SPPS methods are known in the art and may be performed with orthogonal protecting groups. In some embodiments, peptides of the present disclosure may be synthesized via SPPS with Fmoc chemistry and/or Boc chemistry. Synthesized peptides may be cleaved from solid supports using standard techniques.

Peptides may be purified via chromatography [e.g., size exclusion chromatography (SEC) and/or high performance liquid chromatography (HPLC)]. HPLC may include reverse phase HPLC (RP-HPLC). Peptides may be freeze-dried after purification. Purified peptides may be obtained as pure peptide or as a peptide salt. Residual salts making up peptide salts may include, but are not limited to, trifluoroacetic acid (TFA), acetate, and/or hydrochloride. In some embodiments, peptides of the present disclosure are obtained as peptide salts. The peptide salts may be peptide salts with TFA. Residual salts may be removed from purified peptides according to known methods (e.g., through use of desalting columns).

In some embodiments, cyclic C5 inhibitor polypeptides of the present disclosure are formed using a lactam moiety. Such cyclic polypeptides may be formed, for example, by synthesis on a solid support Wang resin using standard Fmoc chemistry. In some cases, Fmoc-ASP(allyl)-OH and Fmoc-LYS(alloc)-OH are incorporated into polypeptides to serve as precursor monomers for lactam bridge formation.

C5 inhibitor polypeptides of the present disclosure may be peptidomimetics. A “peptidomimetic” or “polypeptide mimetic” is a polypeptide in which the molecule contains structural elements that are not found in natural polypeptides (i.e., polypeptides comprised of only the 20 proteinogenic amino acids). In some embodiments, peptidomimetics are capable of recapitulating or mimicking the biological action(s) of a natural peptide. A peptidomimetic may differ in many ways from natural polypeptides, for example through changes in backbone structure or through the presence of amino acids that do not occur in nature. In some cases, peptidomimetics may include amino acids with side chains that are not found among the known 20 proteinogenic amino acids; non-polypeptide-based bridging moieties used to effect cyclization between the ends or internal portions of the molecule; substitutions of the amide bond hydrogen moiety by methyl groups (N-methylation) or other alkyl groups; replacement of a peptide bond with a chemical group or bond that is resistant to chemical or enzymatic treatments; N- and C-terminal modifications; and/or conjugation with a non-peptidic extension (such as polyethylene glycol, lipids, carbohydrates, nucleosides, nucleotides, nucleoside bases, various small molecules, or phosphate or sulfate groups).

As used herein, the term “amino acid” includes the residues of the natural amino acids as well as non-natural amino acids. The 20 natural proteinogenic amino acids are identified and referred to herein by either the one-letter or three-letter designations as follows: aspartic acid (Asp:D), isoleucine threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagine (Asn:N). Naturally occurring amino acids exist in their levorotary (L) stereoisomeric forms. Amino acids referred to herein are L-stereoisomers except where otherwise indicated. The term “amino acid” also includes amino acids bearing a conventional amino protecting group (e.g. acetyl or benzyloxycarbonyl), as well as natural and non-natural amino acids protected at the carboxy terminus (e.g., as a (C1-C6) alkyl, phenyl or benzyl ester or amide; or as an alpha-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc., and documents cited therein, the contents of each of which are herein incorporated by reference in their entirety). Polypeptides and/or polypeptide compositions of the present disclosure may also include modified amino acids.

“Non-natural” amino acids have side chains or other features not present in the 20 naturally-occurring amino acids listed above and include, but are not limited to: N-methyl amino acids, N-alkyl amino acids, alpha, alpha substituted amino acids, beta-amino acids, alpha-hydroxy amino acids, D-amino acids, and other non-natural amino acids known in the art (See, e.g., Josephson et al., (2005) J. Am. Chem. Soc. 127: 11727-11735; Forster, A. C. et al. (2003) Proc. Natl. Acad. Sci. USA 100: 6353-6357; Subtelny et al., (2008) J. Am. Chem. Soc. 130: 6131-6136; Hartman, M. C. T. et al. (2007) PLoS ONE 2:e972; and Hartman et al., (2006) Proc. Natl. Acad. Sci. USA 103:4356-4361). Further non-natural amino acids useful for the optimization of polypeptides and/or polypeptide compositions of the present disclosure include, but are not limited to 1,2,3,4-tetrahydroisoquinoline-1-carboxylic acid, 1-amino-2,3-hydro-1H-indene-1-carboxylic acid, homolysine, homoarginine, homoserine, 2-aminoadipic acid, 3-aminoadipic acid, beta-alanine, aminopropionic acid, 2-aminobutyric acid, 4-aminobutyric acid, 5-aminopentanoic acid, 5-aminohexanoic acid, 6-aminocaproic acid, 2-aminoheptanoic acid, 2-aminoisobutyric acid, 3-aminoisobutyric acid, 2-aminopimelic acid, desmosine, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylpentylglycine, naphthylalanine, ornithine, pentylglycine, thioproline, norvaline, tert-butylglycine, phenylglycine, azatryptophan, 5-azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-azidopentanoic acid (also referred to herein as “X02”), (S)-2-aminohept-6-enoic acid (also referred to herein as “X30”), (S)-2-aminopent-4-ynoic acid (also referred to herein as “X31”), (S)-2-aminopent-4-enoic acid (also referred to herein as “X12”), (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, and (S)-2-amino-3-(1H-indazol-3-yl)propanoic acid, (S)-2-amino-3-(oxazol-2-yl)butanoic acid, (S)-2-amino-3-(oxazol-5-yl) butanoic acid, (S)-2-amino-3-(1,3,4-oxadiazol-2-yl) butanoic acid, (S)-2-amino-3-(1,2,4-oxadiazol-3-yl) butanoic acid, (S)-2-amino-3-(5-fluoro-1H-indazol-3-yl) butanoic acid, and (S)-2-amino-3-(1H-indazol-3-yl) butanoic acid, 2-(2′MeOphenyl)-2-amino acetic acid, tetrahydro 3-isoquinolinecarboxylic acid and stereoisomers thereof (including, but not limited, to D and L isomers).

Additional non-natural amino acids that are useful in the optimization of polypeptides or polypeptide compositions of the present disclosure include but are not limited to fluorinated amino acids wherein one or more carbon bound hydrogen atoms are replaced by fluorine. The number of fluorine atoms included can range from 1 up to and including all of the hydrogen atoms. Examples of such amino acids include but are not limited to 3-fluoroproline, 3,3-difluoroproline, 4-fluoroproline, 4,4-difluoroproline, 3,4-difluroproline, 3,3,4,4-tetrafluoroproline, 4-fluorotryptophan, 5-flurotryptophan, 6-fluorotryptophan, 7-fluorotryptophan, and stereoisomers thereof.

Further non-natural amino acids that are useful in the optimization of polypeptides of the present disclosure include but are not limited to those that are disubstituted at the α-carbon. These include amino acids in which the two substituents on the α-carbon are the same, for example α-amino isobutyric acid, and 2-amino-2-ethyl butanoic acid, as well as those where the substituents are different, for example α-methylphenylglycine and α-methylproline. Further the substituents on the α-carbon may be taken together to form a ring, for example 1-aminocyclopentanecarboxylic acid, 1-aminocyclobutanecarboxylic acid, 1-aminocyclohexanecarboxylic acid, 3-aminotetrahydrofuran-3-carboxylic acid, 3-aminotetrahydropyran-3-carboxylic acid, 4-aminotetrahydropyran-4-carboxylic acid, 3-aminopyrrolidine-3-carboxylic acid, 3-aminopiperidine-3-carboxylic acid, 4-aminopiperidinnne-4-carboxylix acid, and stereoisomers thereof.

Additional non-natural amino acids that are useful in the optimization of polypeptides or polypeptide compositions of the present disclosure include but are not limited to analogs of tryptophan in which the indole ring system is replaced by another 9 or 10 membered bicyclic ring system with 0, 1, 2, 3 or 4 heteroatoms independently selected from N, O, or S. Each ring system may be saturated, partially unsaturated, or fully unsaturated. The ring system may be substituted by 0, 1, 2, 3, or 4 substituents at any substitutable atom. Each substituent may be independently selected from H, F, Cl, Br, CN, COOR, CONRR′, oxo, OR, NRR′. Each R and R′ may be independently selected from H, C1-C20 alkyl, or C1-C20 alkyl-O—C1-20 alkyl.

In some embodiments, analogs of tryptophan (also referred to herein as “tryptophan analogs”) may be useful in the optimization of polypeptides or polypeptide compositions of the present disclosure. Tryptophan analogs may include, but are not limited to, 5-fluorotryptophan [(5-F)W], 5-methyl-O-tryptophan [(5-MeO)W], 1-methyltryptophan [(1-Me-W) or (1-Me)W], D-tryptophan (D-Trp), azatryptophan (including, but not limited to 4-azatryptophan, 7-azatryptophan and 5-azatryptophan) 5-chlorotryptophan, 4-fluorotryptophan, 6-fluorotryptophan, 7-fluorotryptophan, and stereoisomers thereof. Except where indicated to the contrary, the term “azatryptophan” and its abbreviation, “azaTrp,” as used herein, refer to 7-azatryptophan.

Modified amino acid residues useful for the optimization of polypeptides and/or polypeptide compositions of the present disclosure include, but are not limited to those which are chemically blocked (reversibly or irreversibly); chemically modified on their N-terminal amino group or their side chain groups; chemically modified in the amide backbone, as for example, N-methylated, D (non-natural amino acids) and L (natural amino acids) stereoisomers; or residues wherein the side chain functional groups are chemically modified to another functional group. In some embodiments, modified amino acids include without limitation, methionine sulfoxide; methionine sulfone; aspartic acid-(beta-methyl ester), a modified amino acid of aspartic acid; N-ethylglycine, a modified amino acid of glycine; alanine carboxamide; and/or a modified amino acid of alanine. Non-natural amino acids may be purchased from Sigma-Aldrich (St. Louis, Mo.), Bachem (Torrance, Calif.) or other suppliers. Non-natural amino acids may further include any of those listed in Table 2 of US patent publication US 2011/0172126, the contents of which are incorporated herein by reference in their entirety.

The present disclosure contemplates variants and derivatives of polypeptides presented herein. These include substitutional, insertional, deletional, and covalent variants and derivatives. As used herein, the term “derivative” is used synonymously with the term “variant” and refers to a molecule that has been modified or changed in any way relative to a reference molecule or starting molecule.

Polypeptides of the present disclosure may include any of the following components, features, or moieties, for which abbreviations used herein include: “Ac” and “NH2” indicate acetyl and amidated termini, respectively; “Nvl” stands for norvaline; “Phg” stands for phenylglycine; “Tbg” stands for tert-butylglycine; “Chg” stands for cyclohexylglycine; “(N-Me)X” stands for the N-methylated form of the amino acid indicated by the letter or three letter amino acid code in place of variable “X” written as N-methyl-X [e.g. (N-Me)D or (N-Me)Asp stand for the N-methylated form of aspartic acid or N-methyl-aspartic acid]; “azaTrp” stands for azatryptophan; “(4-F)Phe” stands for 4-fluorophenylalanine; “Tyr(OMe)” stands for O-methyl tyrosine, “Aib” stands for amino isobutyric acid; “(homo)F” or “(homo)Phe” stands for homophenylalanine; “(2-OMe)Phg” refers to 2-O-methylphenylglycine; “(5-F)W” refers to 5-fluorotryptophan; “D-X” refers to the D-stereoisomer of the given amino acid “X” [e.g. (D-Chg) stands for D-cyclohexylglycine]; “(5-MeO)W” refers to 5-methyl-O-tryptophan; “homoC” refers to homocysteine; “(1-Me-W)” or “(1-Me)W” refers to 1-methyltryptophan; “Nle” refers to norleucine; “Tiq” refers to a tetrahydroisoquinoline residue; “Asp(T)” refers to (S)-2-amino-3-(1H-tetrazol-5-yl)propanoic acid; “(3-Cl-Phe)” refers to 3-chlorophenylalanine; “[(N-Me-4-F)Phe]” or “(N-Me-4-F)Phe” refers to N-methyl-4-fluorophenylalanine; “(m-Cl-homo)Phe” refers to meta-chloro homophenylalanine; “(des-amino)C” refers to 3-thiopropionic acid; “(alpha-methyl)D” refers to alpha-methyl L-aspartic acid; “2Nal” refers to 2-naphthylalanine; “(3-aminomethyl)Phe” refers to 3-aminomethyl-L-phenyalanine; “Cle” refers to cycloleucine; “Ac-Pyran” refers to 4-amino-tetrahydro-pyran-4-carboxylic acid; “(Lys-C16)” refers to N-ε-palmitoyl lysine; “(Lys-C12)” refers to N-ε-lauryl lysine; “(Lys-C10)” refers to N-ε-capryl lysine; “(Lys-C8)” refers to N-ε-caprylic lysine; “[xXylyl(y,z)]” refers to the xylyl bridging moiety between two thiol containing amino acids where x may be m, p or o to indicate the use of meta-, para- or ortho-dibromoxylenes (respectively) to generate bridging moieties and the numerical identifiers, y and z, place the amino acid position within the polypeptide of the amino acids participating in the cyclization; “[cyclo(y,z)]” refers to the formation of a bond between two amino acid residues where the numerical identifiers, y and z, place the position of the residues participating in the bond; “[cyclo-olefinyl(y,z)]” refers to the formation of a bond between two amino acid residues by olefin metathesis where the numerical identifiers, y and z, place the position of the residues participating in the bond; “[cyclo-thioalkyl(y,z)]” refers to the formation of a thioether bond between two amino acid residues where the numerical identifiers, y and z, place the position of the residues participating in the bond; “[cyclo-triazolyl(y,z)]” refers to the formation of a triazole ring between two amino acid residues where the numerical identifiers, y and z, place the position of the residues participating in the bond. “B20” refers to N-ε-(PEG2-γ-glutamic acid-N-α-octadecanedioic acid) lysine [also known as (1S,28S)-1-amino-7,16,25,30-tetraoxo-9,12,18,21-tetraoxa-6,15,24,29-tetraazahexatetracontane-1,28,46-tricarboxylic acid.]

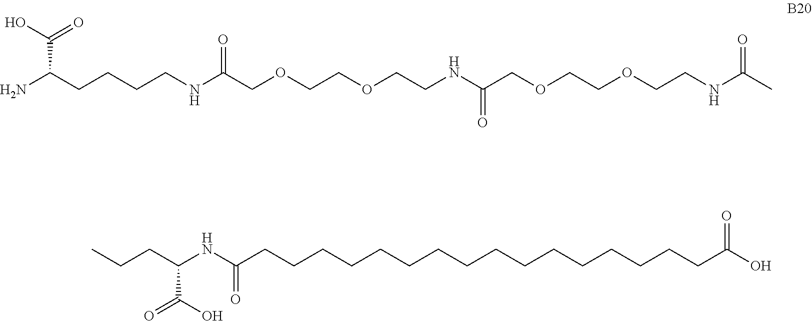

“B28” refers to N-ε-(PEG24-γ-glutamic acid-N-α-hexadecanoyl)lysine.

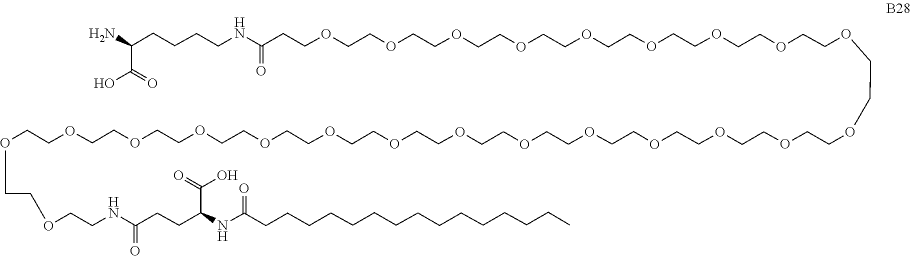 [SMILES: CCCCCCCCCCCCCCCC(=O)N[C@@H](CCC(=O)NCCOCCOCCOCCOCCOCCOCCOCCOCCOCCOCCOCCOCCOCCOCCOCCOCCOCCOCCOCCOCCOCCOCCOCCOCCC(=O)NCCCC[C@H](N)C(=O)O)C(=O)O]

“K14” refers to N-ε-1-(4,4-dimethyl-2,6-dioxocyclohex-1-ylidene)-3-methylbutyl-L-lysine. All other symbols refer to the standard one-letter amino acid code.

Some C5 inhibitor polypeptides include from about 5 amino acids to about 10 amino acids, from about 6 amino acids to about 12 amino acids, from about 7 amino acids to about 14 amino acids, from about 8 amino acids to about 16 amino acids, from about 10 amino acids to about 18 amino acids, from about 12 amino acids to about 24 amino acids, or from about 15 amino acids to about 30 amino acids. In some cases, C5 inhibitor polypeptides include at least 30 amino acids.

Some C5 inhibitors of the present disclosure include a C-terminal lipid moiety. Such lipid moieties may include fatty acyl groups (e.g., saturated or unsaturated fatty acyl groups). In some cases, the fatty acyl group may be a palmitoyl group.

C5 inhibitors having fatty acyl groups may include one or more molecular linkers joining the fatty acids to the peptide. Such molecular linkers may include amino acid residues. In some cases, L-γ glutamic acid residues may be used as molecular linkers. In some cases, molecular linkers may include one or more polyethylene glycol (PEG) linkers. PEG linkers of the present disclosure may include from about 1 to about 5, from about 2 to about 10, from about 4 to about 20, from about 6 to about 24, from about 8 to about 32, or at least 32 PEG units.

C5 inhibitors disclosed herein may have molecular weights of from about 200 g/mol to about 600 g/mol, from about 500 g/mol to about 2000 g/mol, from about 1000 g/mol to about 5000 g/mol, from about 3000 g/mol to about 4000 g/mol, from about 2500 g/mol to about 7500 g/mol, from about 5000 g/mol to about 10000 g/mol, or at least 10000 g/mol.

In some embodiments, C5 inhibitor polypeptides of the present disclosure include zilucoplan. The core amino acid sequence of zilucoplan ([cyclo(1,6)]Ac—K—V-E-R—F-D-(N-Me)D-Tbg-Y-azaTrp-E-Y—P-Chg-K; SEQ ID NO: 1) includes 15 amino acids (all L-amino acids), including 4 non-natural amino acids [N-methyl-aspartic acid or “(N-Me)D”, tert-butylglycine or “Tbg”, 7-azatryptophan or “azaTrp”, and cyclohexylglycine or “Chg”]; a lactam bridge between K1 and D6 of the polypeptide sequence; and a C-terminal lysine reside with a modified side chain, forming a N-ε-(PEG24-γ-glutamic acid-N-α-hexadecanoyl)lysine residue (also referred to herein as “B28”). The C-terminal lysine side chain modification includes a polyethyleneglycol (PEG) spacer (PEG24), with the PEG24 being attached to an L-γ glutamic acid residue that is derivatized with a palmitoyl group.

The free acid form of zilucoplan has a molecular formula of C172H278N24055, a molecular weight of 3562.23 Daltons (Da), and an exact mass of 3559.97 amu (see CAS Number 1841136-73-9). The tetra sodium form of zilucoplan has a molecular formula of C₁₇₂H₂₇₈N₂₄O₅₅Na₄. The chemical structure of sodium salt form of zilucoplan is shown in structure I:

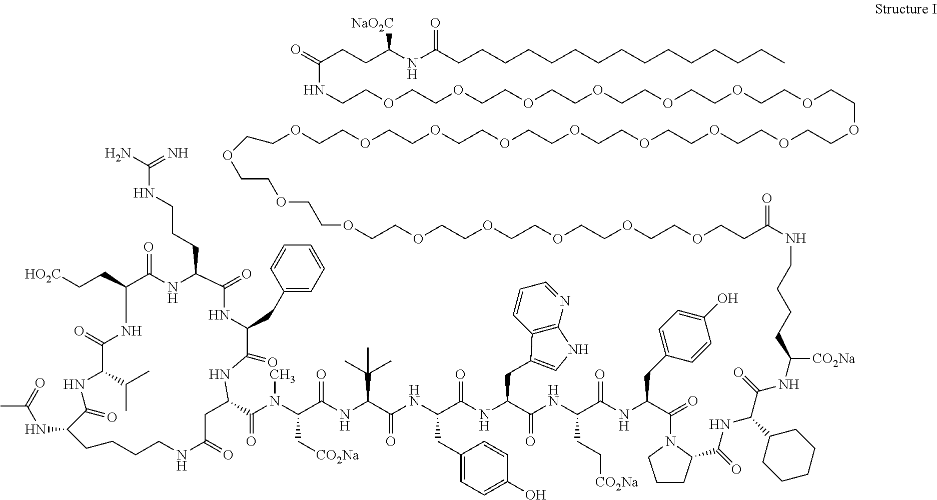

The four sodium ions in the structure are shown associated with designated carboxylates, but they may be associated with any of the acidic groups in the molecule. The zilucoplan drug substance is typically provided as the sodium salt form and is lyophilized. The free base form of zilucoplan or any pharmaceutically acceptable salt of zilucoplan are encompassed by the term “zilucoplan”.

In some embodiments, the present disclosure includes variants of zilucoplan. In some zilucoplan variants, the C-terminal lysine side chain moiety may be altered. In some cases, the PEG24 spacer (having 24 PEG subunits) of the C-terminal lysine side chain moiety may include fewer or additional PEG subunits. In other cases, the palmitoyl group of the C-terminal lysine side chain moiety may be substituted with another saturated or unsaturated fatty acid. In further cases, the L-γ glutamic acid linker of the C-terminal lysine side chain moiety (between PEG and acyl groups) may be substituted with an alternative amino acid or non-amino acid linker.

In some embodiments, C5 inhibitors may include active metabolites or variants of zilucoplan. Metabolites may include w-hydroxylation of the palmitoyl tail. Such variants may be synthesized or may be formed by hydroxylation of a zilucoplan precursor.

In some embodiments, zilucoplan variants may include modifications to the core polypeptide sequence in zilucoplan that may be used in combination with one or more of the cyclic or C-terminal lysine side chain moiety features of zilucoplan. Such variants may have at least 50%, at least 55%, at least 65%, at least 70%, at least 80%, at least 85%, at least 90%, or at least 95% sequence identity to the core polypeptide sequence of (SEQ ID NO: 1).

In some cases, zilucoplan variants may be cyclized by forming lactam bridges between amino acids other than those used in zilucoplan.

In some embodiments, C5 inhibitors of the present disclosure may include any of those listed in Table 1 of United States Publication Number US 2017/0137468, the contents of which are herein incorporated by reference in their entirety.

C5 inhibitors of the present disclosure may be developed or modified to achieve specific binding characteristics. Inhibitor binding may be assessed by determining rates of association and/or dissociation with a particular target. In some cases, compounds demonstrate strong and rapid association with a target combined with a slow rate of dissociation. In some embodiments, C5 inhibitors of the present disclosure demonstrate strong and rapid association with C5. Such inhibitors may further demonstrate slow rates of dissociation with C5.

C5 protein-binding C5 inhibitors disclosed herein, may bind to C5 complement protein with an equilibrium dissociation constant (K_{D}) of from about 0.001 nM to about 0.01 nM, from about 0.005 nM to about 0.05 nM, from about 0.01 nM to about 0.1 nM, from about 0.05 nM to about 0.5 nM, from about 0.1 nM to about 1.0 nM, from about 0.5 nM to about 5.0 nM, from about 2 nM to about 10 nM, from about 8 nM to about 20 nM, from about 15 nM to about 45 nM, from about 30 nM to about 60 nM, from about 40 nM to about 80 nM, from about 50 nM to about 100 nM, from about 75 nM to about 150 nM, from about 100 nM to about 500 nM, from about 200 nM to about 800 nM, from about 400 nM to about 1,000 nM or at least 1,000 nM.

In some embodiments, C5 inhibitors of the present disclosure block the formation or generation of C5a from C5. In some case, formation or generation of C5a is blocked following activation of the alternative pathway of complement activation. In some cases, C5 inhibitors of the present disclosure block the formation of the membrane attack complex (MAC). Such MAC formation inhibition may be due to C5 inhibitor binding to C5b subunits. C5 inhibitor binding to C5b subunits may prevent C6 binding, resulting in blockage of MAC formation. In some embodiments, this MAC formation inhibition occurs after activation of the classical, alternative, or lectin pathways.

C5 inhibitors of the present disclosure may be synthesized using chemical processes. In some cases, such synthesis eliminates risks associated with the manufacture of biological products in mammalian cell lines. In some cases, chemical synthesis may be simpler and more cost-effective than biological production processes.

In some embodiments, C5 inhibitor (e.g., zilucoplan and/or an active metabolite or variant thereof) compositions may be pharmaceutical compositions that include at least one pharmaceutically acceptable excipient. In some embodiments, the pharmaceutically acceptable excipient may include at least one of a salt and a buffering agent. The salt may be sodium chloride. The buffering agent may be sodium phosphate. Sodium chloride may be present at a concentration of from about 0.1 mM to about 1000 mM. In some cases, sodium chloride may be present at a concentration of from about 25 mM to about 100 mM. Sodium phosphate may be present at a concentration of from about 0.1 mM to about 1000 mM. In some cases, sodium phosphate is present at a concentration of from about 10 mM to about 100 mM.

In some embodiments, C5 inhibitor (e.g., zilucoplan and/or an active metabolite or variant thereof) compositions may include from about 0.01 mg/mL to about 4000 mg/mL of a C5 inhibitor. In some cases, C5 inhibitors are present at a concentration of from about 1 mg/mL to about 400 mg/mL.

Zilucoplan binds to C5 and inhibits cleavage of C5 by canonical complement pathway convertases as described in International Publication Number WO2018106859, the contents of which are herein incorporated by reference in their entirety. Zilucoplan additionally binds C5b, preventing the formation of the membrane attack complex induced by non-canonical cleavage of C5. Zilucoplan binding and inhibitory activities are not affected by the presence of clinically-relevant human C5 polymorphisms (including p.R885>H/C). Unlike eculizumab, an anti-05 monoclonal antibody inhibitor, zilucoplan does not bind to surface-bound C5b-9 or soluble membrane attack complex (sC5b-9).

### Isotopic Variations

Compounds of the present disclosure may include one or more atoms that are isotopes. As used herein, the term “isotope” refers to a chemical element that has one or more additional neutrons. In some embodiments, compounds of the present disclosure may be deuterated. As used herein, the term “deuterated” refers to a substance that has had one or more hydrogen atoms replaced by deuterium isotopes. Deuterium isotopes are isotopes of hydrogen. The nucleus of hydrogen contains one proton while deuterium nuclei contain both a proton and a neutron. Compounds and compositions of the present disclosure may be deuterated in order to change a physical property, such as stability, or to allow for use in diagnostic and experimental applications.

### II. Methods and Uses

In some embodiments, the present disclosure provides methods related to using and evaluating compounds and compositions described herein for therapeutic treatment of various therapeutic indications. Some methods include modulating complement activity using compounds and/or compositions described herein.

### Therapeutic Indications

In some embodiments, methods of the present disclosure include methods of treating therapeutic indications using compounds and/or compositions disclosed herein. As used herein, the term “therapeutic indication” refers to any symptom, condition, disorder, or disease that may be alleviated, stabilized, improved, cured, or otherwise addressed by some form of treatment or other therapeutic intervention (e.g., through complement inhibitor administration). Therapeutic indications may include, but are not limited to, inflammatory indications, wounds, injuries, autoimmune indications, vascular indications, neurological indications, kidney-related indications, ocular indications, cardiovascular indications, pulmonary indications, and pregnancy-related indications. Therapeutic indications associated with complement activity and/or dysfunction are referred to herein as “complement-related indications.” In some embodiments, methods of the present disclosure may include treating complement-related indications by administering compounds and/or compositions disclosed herein (e.g., complement inhibitor compounds).

In some embodiments, complement inhibitor compounds may be useful in the treatment of complement-related indications where complement activation leads to progression of a disease, disorder and/or condition. Such complement-related indications may include, but are not limited to inflammatory indications, wounds, injuries, autoimmune indications, vascular indications, neurological indications, kidney-related indications, ocular indications, cardiovascular indications, pulmonary indications, and pregnancy-related indications. Complement-related indications may include, but are not limited to, any of those listed in U.S. Pat. No. 10,106,579, the contents of which are herein incorporated by reference in their entirety.

Complement inhibitor compounds and compositions may be useful in the treatment of infectious diseases, disorders and/or conditions, for example, in a subject having an infection. In some embodiments, subjects having an infection or that are at risk of developing sepsis or a septic syndrome may be treated with complement inhibitors described herein. In some cases, complement inhibitor compounds may be used in the treatment of sepsis.

Complement inhibitor compounds and compositions may also be administered to improve the outcome of clinical procedures wherein complement inhibition is desired. Such procedures may include, but are not limited to grafting, transplantation, implantation, catheterization, intubation and the like. In some embodiments, complement inhibitor compounds and compositions are used to coat devices, materials and/or biomaterials used in such procedures. In some embodiments, the inner surface of a tube may be coated with compounds and compositions to prevent complement activation within a bodily fluid that passes through the tube, either in vivo or ex vivo, e.g., extracorporeal shunting, e.g., dialysis and cardiac bypass.

As used herein the terms “treat,” “treatment,” and the like, refer to relief from or alleviation of pathological processes. In the context of the present disclosure insofar as it relates to any of the other conditions recited herein below, the terms “treat,” “treatment,” and the like mean to relieve or alleviate at least one symptom associated with such condition, or to slow or reverse the progression or anticipated progression of such condition.

By “lower” or “reduce” in the context of a disease marker or symptom is meant a significant decrease in such a level, often statistically significant. The decrease may be, for example, at least 10%, at least 20%, at least 30%, at least 40% or more, and is preferably down to a level accepted as within the range of normal for an individual without such a disorder.

By “increase” or “raise” in the context of a disease marker or symptom is meant a significant rise in such level, often statistically significant. The increase may be, for example, at least 10%, at least 20%, at least 30%, at least 40% or more, and is preferably up to a level accepted as within the range of normal for an individual without such disorder.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of a polypeptide or pharmaceutical composition thereof, “effective against” a disease or disorder indicates that administration in a clinically appropriate manner results in a beneficial effect for at least a fraction of patients, such as an improvement of symptoms, a cure, a reduction in disease load, reduction in tumor mass or cell numbers, extension of life, improvement in quality of life, a reduction in the need for blood transfusions or other effect generally recognized as positive by medical doctors familiar with treating the particular type of disease or disorder.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more may be indicative of effective treatment. Efficacy for a given compound or composition may also be judged using an experimental animal model for the given disease as known in the art. When using an experimental animal model, efficacy of treatment is evidenced when a statistically significant modulation in a marker or symptom is observed.

Compounds of the present disclosure and additional therapeutic agents can be administered in combination. Such combinations may be in the same composition, or the additional therapeutic agents can be administered as part of a separate composition or by another method described herein.

In some embodiments, the present disclosure provides methods of inhibiting C5 activity in a tissue by contacting the tissue with a tissue-penetrating C5 inhibitor. As used herein, the term “tissue-penetrating” refers to a property characterized by tissue permeability. Agents with enhanced tissue-penetration may demonstrate better distribution in tissues when compared to agents with less or no tissue-penetration. Tissue penetration may be assessed by ability to cross basement membranes. As used herein, the term “basement membrane” refers to an extracellular matrix (ECM) protein layer separating endothelial cells from underlying tissues. Tissue penetration assessments may be done in vivo or in vitro and may include the use of basement membrane models. Such models may include measuring compound diffusion across artificial basement membranes. Such models may include the use of upper and lower reservoirs separated by an artificial basement membrane. Artificial basement membranes may include any of the ECM gel membranes described in Arends, F. et al. 2016. IntechOpen, DOI: 10.5772/62519, the contents of which are herein incorporated by reference in their entirety. ECM gel membranes may be prepared to include matrix components mimicking those found in the basal lamina of neuromuscular junctions. In some models, compounds being tested are introduced to upper reservoirs and compound diffusion is detected in lower reservoirs.

Tissue penetration assessment may include visual assessments e.g., through use of fluorescent labels to visualize analyte movement across basement membranes. Some assessments may include biochemical analysis of samples obtained from the penetrated side of a basement membrane.

In some embodiments, compound permeability may be determined using quantitative whole body analysis (QWBA). QWBA is a form of analysis that uses radiography to assess distribution of radiolabeled analytes. In some embodiments, radiolabeled compounds are administered to subjects and tissue distribution of the compounds is analyzed over time.

Tissue-penetrating C5 inhibitors may be polypeptides. Tissue-penetrating C5 inhibitors may include zilucoplan (see International Publication Number WO2020086506, the contents of which are herein incorporated by reference in their entirety). Contacting tissues with the tissue-penetrating C5 inhibitors may include administering tissue-penetrating C5 inhibitors to tissues as part of a formulation. Such formulations may be administered by subcutaneous injection. Tissue-penetrating C5 inhibitors may be able to penetrate basement membranes. Basement membrane permeability of polypeptide tissue-penetrating C5 inhibitors may be greater than basement membrane permeability of larger proteins, such as antibodies. Such advantages may be due to restrictively large size of proteins and antibodies. Zilucoplan basement membrane permeability may be from about 3-fold to about 5-fold greater than basement membrane permeability of eculizumab, offering advantages over eculizumab for inhibiting C5 activity in tissues and treating related complement-related indications. In some embodiments, zilucoplan permeability enhances distribution in one or more of lung, heart, muscle, small intestine, large intestine, spleen, liver, bone, stomach, lymph node, fat, brain, pancreas, testes, and thymus, in comparison to eculizumab.

### Inflammatory Indications

Therapeutic indications that may be addressed with compounds and/or compositions of the present disclosure may include inflammatory indications. As used herein, the term “inflammatory indication” refers to therapeutic indications that involve immune system activation. Inflammatory indications may include complement-related indications. Inflammation may be upregulated during the proteolytic cascade of the complement system. Although inflammation may have beneficial effects, excess inflammation may lead to a variety of pathologies (Markiewski et al. 2007. Am J Pathol. 17: 715-27). In some embodiments, complement inhibitor compounds and compositions of the present disclosure may be used to treat, prevent, or delay development of inflammatory indications.

Inflammatory indications may include, but are not limited to, Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Acute antibody-mediated rejection following organ transplantation, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Autoimmune urticaria, Axonal & neuronal neuropathies, Bacterial sepsis and septic shock, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal osteomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Diabetes Type I, Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia (including atypical hemolytic uremic syndrome and plasma therapy-resistant atypical hemolytic-uremic syndrome), Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Insulin-dependent diabetes (type1), Interstitial cystitis, Juvenile arthritis, Juvenile diabetes, Kawasaki syndrome, Lambert-Eaton syndrome, Large vessel vasculopathy, Leukocytoclastic vasculitis, Lichen planus, Lichen sclerosus, Ligneous conjunctivitis, Linear IgA disease (LAD), Lupus (SLE), Lyme disease, Meniere's disease, Microscopic polyangiitis, Mixed connective tissue disease (MCTD), Mooren's ulcer, Mucha-Habermann disease, Multiple endocrine neoplasia syndromes, Multiple sclerosis, Multifocal motor neuropathy, Myositis, Myasthenia gravis, Narcolepsy, Neuromyelitis optica (Devic's), Neutropenia, Ocular cicatricial pemphigoid, Optic neuritis, Osteoarthritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polyendocrinopathies, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic Pulmonary fibrosis, Pyoderma gangrenosum, Pure red cell aplasia, Raynauds phenomenon, Reactive arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Shiga-Toxin producing Escherichia coli Hemolytic-Uremic Syndrome (STEC-HUS), Sjogren's syndrome, Small vessel vasculopathy, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Tubular autoimmune disorder, Ulcerative colitis, Undifferentiated connective tissue disease (UCTD), Uveitis, Vesiculobullous dermatosis, Vasculitis, Vitiligo, and Wegener's granulomatosis (also known as Granulomatosis with Polyangiitis (GPA)).

### Neuromuscular Inflammatory Indications

Therapeutic indications that may be addressed with compounds and/or compositions of the present disclosure may include neuromuscular inflammatory indications. As used herein, the term “neuromuscular inflammatory indication” refers to inflammatory indications that involve components of the nervous system, muscular system, and areas where these two systems overlap or integrate. Neuromuscular inflammatory indications may include complement-related indications. Neuromuscular inflammation may be upregulated during the proteolytic cascade of the complement system. Although inflammation may have beneficial effects, excess inflammation may lead to a variety of pathologies (Markiewski et al. 2007. Am J Pathol. 17: 715-27). In some embodiments, complement inhibitor compounds and compositions of the present disclosure may be used to treat, prevent, or delay development of neuromuscular inflammatory indications.

In some embodiments, the present disclosure provides methods of treating neuromuscular inflammatory indications by administering complement inhibitors to subjects. Neuromuscular inflammatory indications may include amyotrophic lateral sclerosis. Methods of treating neuromuscular inflammatory indications may include administering complement inhibitors. Such methods may include complement inhibitor compounds (e.g., C5 inhibitor compounds, e.g., zilucoplan) and compositions disclosed herein. In some embodiments, complement inhibitor compounds are administered in combination with other therapeutic agents and/or therapeutic regimen.

Complement inhibitors used to treat neuroinflammatory indications may include zilucoplan. Zilucoplan may be administered at a dose of from about 0.1 mg/kg to about 0.3 mg/kg. Zilucoplan may be administered daily. Zilucoplan may be administered by subcutaneous injection. The subcutaneous injection may include the use of an autoinjection device. Autoinjection (synonymous with “self-injection”) devices may include BD ULTRASAFE PLUS™ autoadministration devices (BD, Franklin Lakes, N.J.).

### Amyotrophic Lateral Sclerosis (ALS)

Neuroinflammatory indications that may be addressed with compounds and/or compositions of the present disclosure may include ALS. ALS, also referred to as Lou Gehrig's disease, is a progressive neurodegenerative disease with upper and lower motor neuron loss in the primary motor cortex, brainstem, and spinal cord. Cortical and spinal cord motor neurons normally transmit signals from brain to muscles during voluntary movement. Neuronal loss disrupts these signals resulting in gradual weakening and/or muscle atrophy and disruption of voluntary movement capability. In addition to persistent weakness, patients also experience painful muscle cramping and limb spasticity. Affected individuals eventually lose muscle strength, mobility, and critical neuromotor functions (ability to speak, eat, swallow, and breathe). With time, patients typically require breathing assistance and most die from respiratory failure within 2-5 years of diagnosis. Only 5-10% of cases are thought to be familial, while 90-95% of cases are sporadic.

Approximately 20 gene mutations have been identified that lead to familial cases. These include, but are not limited to, Cu²⁺/Zn²⁺ superoxide dismutase (SOD1), TAR DNA binding protein-43 (TDP-43), Fused in Sarcoma/Translocated in Sarcoma (FUS), Angiogenin (ANG), Ataxin-2 (ATXN2), valosin containing protein (VCP), Optineurin (OPTN), and noncoding GGGGCC hexanucleotide repeat expansion in chromosome 9 open reading frame 72 (C9ORF72). In some cases, patients with ALS also develop frontotemporal dementia (FTD-ALS).

ALS diagnosis may be carried out according to revised El Escorial criteria as described in Brooks R B, et al. Amyotroph Lateral Scler Other Motor Neuron Disord. 2000 December; 1(5):293-9 and Ludolph, A. et al., ALS and FD, 2015. Early Online: 1-2. According to such analysis, subject upper motor neuron (UMN) and lower motor neuron (LMN) impairment is assessed in bulbar, cervical, thoracic, and lumbosacral regions of the body. UMN assessment includes analysis of spasticity and hyperreflexivity. LMN assessment includes assessment of weakness, atrophy, and fasciculation. Subjects with signs of UMN and LMN impairment in at least three regions tested are diagnosed as having “definite” ALS. Subjects with signs of UMN and LMN impairment in at least two regions tested are diagnosed as having “probable” ALS. Subjects with UMN and LMN signs in one region with evidence by electromyography (EMG) of LMN involvement in another region are characterized as having “laboratory results-supported probable” ALS. Subjects with signs of UMN and LMN impairment in one region tested or have UMN impairment alone in two or more regions are characterized as having “possible” ALS. Subjects with signs of LMN impairment alone in two or more regions tested are characterized as having “suspected” ALS.

Current treatments are not curative and are used to temporarily alleviate or slow the progression of symptoms. Such treatments include Riluzole, which is used to reduce pathological glutamate response. Life-span expansion reported with this treatment has not exceeded three months and only with early stage treatment (Bensimon G et al., J Neurol. 2002, 249, 609-615). Other treatments include Radicava (edaravone), which has been shown to slow disease progression, but not cure the disease.

Complement cascade activation is evident in peripheral and central ALS pathophysiology and is closely associated with ongoing neuroinflammatory processes. Complement activation may temporally precede motor end plate degeneration in the periphery. Complement inhibitor use may be used to block or slow this process. Recent studies have identified MAC deposition in spinal and cerebral neuronal cells, glial cells, and neuromuscular junctions of ALS patients. These cells produce complement proteins C1q and C4 involved in classical pathway activation. C5a receptor (C5aR1) upregulation has also been shown in motorneurons from ALS postmortem donors and SOD1G93A transgenic animals used in ALS disease models. Further, C5aR1 knockout and inhibition have been shown to prolong animal survival in ALS disease models.

In some embodiments, methods of the present disclosure include methods of treating ALS by administering complement inhibitors. Such complement inhibitors may include compounds (e.g., C5 inhibitor compounds, e.g., zilucoplan) and compositions disclosed herein. ALS treatment with complement inhibitors according to the present disclosure may be carried out to reduce, prevent, stabilize, and/or reverse one or more effect of ALS experienced by a subject having or suspected of having ALS. In some embodiments, treatment methods of the present disclosure include administering complement inhibitors in combination with other therapeutic agents and/or therapeutic regimen.

Complement inhibitors used to treat ALS may include C5 inhibitors. Such C5 inhibitors may include zilucoplan. Zilucoplan may be administered at a dose of from about 0.1 mg/kg to about 0.3 mg/kg. Zilucoplan may be administered daily. Zilucoplan may be administered by subcutaneous injection. The subcutaneous injection may include the use of an autoinjection device. Autoinjection devices may include BD ULTRASAFE PLUS™ autoadministration devices (BD, Franklin Lakes, N.J.).

Methods of treating ALS according to the present disclosure may include assessments of treatment efficacy and/or other results of treatment. Such assessments may be carried out before, during, and/or after treatment. Assessments may be carried out to monitor changes in patient symptoms and/or neuromotor functions. Assessments may be carried out during clinical visits or at home. Some assessments may be carried out by subjects undergoing treatment, referred to herein as “self-assessments.” Self-assessments may be carried out with the use of a smart device (e.g., smart watch, mobile phone, computer, etc.). Measures of treatment efficacy may include, but are not limited to, assessment of ALS Functional Rating Scale—Revised (ALSFRS-R); assessment of vital capacity; assessment by hand-held dynamometry (HHD); assessment by bilateral hand grip; and voice analysis.

In some embodiments, assessments carried out with ALS treatment methods include physical and/or neurological examinations. Some assessments may include vital sign assessments. Some assessments may include height and weight assessments. Some assessments may include clinical laboratory assessments. Some assessments may include Edinburgh Cognitive and Behavioral ALS Screen (ECAS). Some assessments may include Columbia Suicide Severity Rating Scale (C-SSRS).

In some embodiments, assessments carried out with ALS treatment methods include collection of one or more samples from subjects receiving treatment. Such subject samples may include biological samples. Samples may include, but are not limited to, blood samples, plasma samples, serum samples, urine samples, spinal fluid samples, or cerebrospinal fluid (CSF) samples. Some subject samples may include DNA. In some embodiments, subject samples are analyzed for one or more biomarkers. Biomarkers may include biomarkers of neurofilament processing and/or axonal neurodegeneration (referred to herein as “neuroaxonal degeneration biomarkers”). Such biomarkers may include, but are not limited to, phosphorylated neurofilament heavy chain (pNfH) and neurofilament light chain (NfL). Biomarkers may include neurotrophin receptor p75 extracellular domain (p75ECD). In some embodiments, biomarkers include biomarkers of neuroinflammation (referred to herein as “neuroinflammation biomarkers”). Neuroinflammation biomarkers may include, but are not limited to, monocyte chemoattractant protein-1 (MCP-1), chitotriosidase-1 (CHIT1) and chitinase-3-like protein 1 (YKL-40). Biomarkers may include complement component C5 or biomarkers of complement activity (referred to herein as “complement activity biomarkers”). Complement activity biomarkers may include cleavage products of C5. Complement activity biomarkers may include, but are not limited to, C5a, C5b-9, soluble C5b-9 (sC5b-9), C5b6 complex, C3a, C4a, Ba, Bb, Factor I, and Factor H.

Biomarkers may be detected in subjects or biological samples obtained from subjects according to standard methods. In some embodiments, biomarkers are detected by immunoassay. As used herein, the term “immunoassay” refers to any form of scientific analysis utilizing antibodies (e.g., for binding and/or detecting antibody-specific targets). Immunoassays may include enzyme-linked immunoassays (ELISAs). Immunoassays may be conducted with solid (e.g., using a fixed surface, plate or well) or solution-based (e.g., using beads or other antibody-coated particles in solution) platforms. In some embodiments, biomarkers are detected using surface plasmon resonance (SPR) based platforms. In some embodiments, complement activity biomarker analysis may be carried out using commercial assays or kits (e.g., single or multi-plex analysis assays from Quidel Corporation, San Diego, Calif.).

In some embodiments, subject biomarkers assessments are used to prepare an ALS biomarker profile. As used herein, the term “ALS biomarker profile” refers to a set of biomarker assessments, wherein the biomarkers are related to ALS (e.g., due to correlation with disease or disease outcome). ALS biomarker profiles may be used to assess disease severity. Changes in ALS biomarker profiles over time may be used to monitor changes in disease state (i.e., stabilization, improvement, or decline). In some embodiments, stable ALS biomarker profile (i.e., no change over time) may be used to indicate no change in disease state. In some embodiments, change in ALS biomarker profile may indicate improving or declining disease state. In some embodiments, change in ALS biomarker profile over time may be used to determine rate of improving or declining disease state.

In some embodiments, subjects exhibit elevated ALS disease severity and/or ALS disease severity that is increasing at a specific rate prior to complement inhibitor treatment. Treatment of such subjects with complement inhibitors (e.g., zilucoplan) according to the present disclosure may stabilize or reduce ALS disease severity and/or reduce a specific rate at which subject ALS disease severity is increasing. Change in subject ALS disease severity may be measured by ALSFRS-R.

In some embodiments, subjects exhibit decreased respiratory function and/or respiratory function that is declining at a specific rate prior to complement inhibitor treatment. Treatment of such subjects with complement inhibitors (e.g., zilucoplan) according to the present disclosure may stabilize or improve subject respiratory function and/or reduce the specific rate at which subject respiratory function is declining. Change in subject respiratory function may be assessed by slow vital capacity (SVC).

In some embodiments, subjects exhibit decreased muscle strength and/or muscle strength that is declining at a specific rate prior to complement inhibitor treatment. Treatment of such subjects with complement inhibitors (e.g., zilucoplan) according to the present disclosure may stabilize or improve subject muscle strength and/or reduce the specific rate at which subject muscle strength is declining. Change in subject muscle strength may be assessed by hand held dynamometry (HHD).

In some embodiments, subjects exhibit decreased speech capability and/or speech capability that is declining at a specific rate prior to complement inhibitor treatment. Treatment of such subjects with complement inhibitors (e.g., zilucoplan) according to the present disclosure may stabilize or improve subject speech capability and/or reduce the specific rate at which subject speech capability is declining. Subject speech capability may be assessed by assessment of one or more of articulatory precision, speaking rate, percent of speech signal voiced, phonation length, consonant length, and nasality.

In some embodiments, methods of the present disclosure include methods of treating ALS in subjects having or suspected of having myasthenia gravis or generalized myasthenia gravis (referred to herein collectively as “MG”) by administering complement inhibitors. Such complement inhibitors may include compounds (e.g., C5 inhibitor compounds, e.g., zilucoplan) and compositions disclosed herein. Such treatments may be carried out to reduce, prevent, stabilize, and/or reverse one or more effect of ALS and/or MG experienced by a subject. Subjects having or suspected of having MG may be identified by positive testing for at least one autoantibody. The at least one autoantibody may include one or more of anti-acetylcholine receptor antibody, anti-LDL receptor related protein 4 (LRP4), anti-agrin, and anti-muscle associated receptor tyrosine kinase. Methods of detecting such autoantibodies in subjects may be carried out according to methods known in the art (e.g., see Rivner et al., 2017. Muscle Nerve. 55(3):430-2; Takahashi et al., 2016. BMC Neurology. 16:229; Tzartos et al., 2014. Ann Clin Transl Neurol. 1(2):80-7; Amador et al., 2016. Neuromuscul Disord. 26(6):342-6; and Mantovani, et al., 2014. J Neuroimmunol. 2014. 276(1-2):213-8, the content of each of which is herein incorporated by reference in its entirety).

In some embodiments, the present disclosure provides compositions that include complement inhibitors for use in methods of treating subjects having or suspected of having ALS. Such compositions may reduce, prevent, stabilize, and/or reverse one or more effects of ALS experienced by subjects. Complement inhibitors included may include C5 inhibitors (e.g., zilucoplan). Subject ALS disease severity may be elevated and/or may be increasing at a specific rate prior to treatment. Treatment with compositions according to the present disclosure may stabilize or reduce subject ALS disease severity and/or reduce the specific rate at which subject ALS disease severity is increasing. Changes in subject ALS disease severity may be measured by ALSFRS-R. Subject respiratory function may be decreased and/or may be declining at a specific rate prior to treatment. Treatment with the compositions may stabilize or improve subject respiratory function and/or reduce the specific rate at which subject respiratory function is declining. Changes in subject respiratory function may be assessed by SVC. Subject muscle strength may be decreased and/or declining at a specific rate prior to treatment. Compositions used in treatments may stabilize or improve subject muscle strength and/or reduce the specific rate at which subject muscle strength is declining. Changes in subject muscle strength may be measured by HHD. Compositions may be used to stabilize or improve subject speech capability and/or rate of subject speech capability decline. Subject speech capability may include one or more of articulatory precision, speaking rate, percent of speech signal voiced, phonation length, consonant length, and nasality. Compositions may be used to stabilize or improve subject ALS biomarker profiles and/or rate of subject ALS biomarker profile change. Subject ALS biomarker profiles may include one or more of pNfH, NfL, C5, and a complement activity biomarker. Complement activity biomarkers may include, but are not limited to, C5a, C5b-9, soluble C5b-9, C3a, C4a, Ba, Bb, Factor I, and Factor H. Complement activity biomarkers may be detected in subject CSF, plasma, and/or serum. Dose and/or regimen of compositions may be adjusted based on complement activity biomarker levels detected in CSF, plasma, and/or serum samples obtained from subjects after prior composition administrations. Complement activity biomarkers detected in CSF, plasma, and/or serum samples obtained from subjects after prior composition administration may include, but are not limited to, C5a, C5b-9, soluble C5b-9, C3a, C4a, Ba, Bb, Factor I, and Factor H. Subjects may have or may be suspected of having myasthenia gravis. Such subjects may be positive for at least one autoantibody (e.g., anti-acetylcholine receptor antibody, anti-LDL receptor related protein 4, anti-agrin, and/or anti-muscle associated receptor tyrosine kinase). Subjects may have impaired blood brain barrier function (e.g., as assessed by subject albumin quotient). Subjects may be determined to have definite ALS or probable ALS based on revised El Escorial criteria.

In some embodiments, methods of treating ALS according to the present disclosure include treating subjects with ALS that have or are suspected of having impaired blood brain barrier function (e.g., as a consequence of elevated complement activity). Assessment of blood brain barrier (BBB) impairment may be carried out by measuring albumin levels in subject CSF and plasma serum. The ratio of CSF albumin level to plasma serum albumin level is referred to as the “albumin quotient” or “albumin-Q.” Increased BBB impairment leads to increased entry of albumin from the plasma compartment into the CSF, which increases the albumin quotient. Albumin quotient values greater than 9 are indicative of BBB impairment. In some embodiments, subjects with other complement-related indications may have BBB impairment (e.g., as assessed by albumin quotient determination). Methods of the present disclosure include methods of treating such subjects with complement inhibitor (e.g., C5 inhibitor) administration. In some embodiments, the complement inhibitor includes zilucoplan.

Methods of treating subjects with ALS according to the present disclosure may include treatment of subjects with definite or probable ALS, as determined by revised El Escorial criteria.

In some embodiments, the present disclosure provides compositions that include complement inhibitors for use in methods of treating subjects having complement-related indications, wherein the subjects have or are suspected of having impaired blood brain barriers. Such compositions may reduce, prevent, stabilize, and/or reverse one or more effects of complement-related indications. Complement inhibitors included may include C5 inhibitors. Complement inhibitors included may include zilucoplan. Complement-related indications may include neuromuscular inflammatory indications. Neuromuscular inflammatory indications may include ALS. Subjects may be determined to have definite ALS or probable ALS based on revised El Escorial criteria. Subjects may be assessed for blood brain barrier impairment by albumin quotient analysis.

In some embodiments, the present disclosure provides methods of diagnosing ALS in subjects by detecting and/or quantifying complement activity associated with such subjects or biological samples from such subjects. Diagnosis may be based on complement activity detected and/or quantified in association with subject biological samples. Complement activity detection and/or quantification may include detecting and/or quantifying complement activity biomarkers. Such complement activity biomarkers may include, but are not limited to, C5a, C5b-9, soluble C5b-9, C3a, C4a, Ba, Bb, Factor I, and Factor H. Complement activity biomarkers may be detected and/or quantified in subject CSF, plasma, and/or serum.

In some embodiments, the present disclosure provides methods of monitoring ALS in subjects by detecting and/or quantifying complement activity associated with such subjects (or biological samples derived therefrom) at initial or earlier time points or time periods and comparing to results of similar analyses from subsequent time points or periods. Such comparisons may be used to monitor ALS in subjects by assessing changes in complement activity between initial/earlier time points or periods and the subsequent time points or periods. Complement activity detection and/or quantification may include detecting and/or quantifying complement activity biomarkers. The complement activity biomarkers may include, but are not limited to C5a, C5b-9, soluble C5b-9, C3a, C4a, Ba, Bb, Factor I, and Factor H. Complement activity biomarkers may be detected and/or quantified in subject CSF, plasma, and/or serum. Methods of monitoring ALS in subjects according to the present disclosure may be carried out with subjects with definite or probable ALS, as determined by revised El Escorial criteria.

In some embodiments, the present disclosure provides methods of stratifying subjects with ALS or suspected of having ALS into two or more groups. Such methods may include detecting and/or quantifying complement activity associated with subjects (or biological sample therefrom) and assigning each subject to at least one of the two or more groups based on complement activity detected and/or quantified. Complement activity detection and/or quantification may include detecting and/or quantifying complement activity biomarkers. Such complement activity biomarker may include, but are not limited to, C5a, C5b-9, soluble C5b-9, C3a, C4a, Ba, Bb, Factor I, and Factor H. Complement activity biomarkers may be detected and/or quantified in subject CSF, plasma, and/or serum. Subjects may be assigned to treatment groups based on CSF, plasma, and/or serum complement activity biomarker levels. As used herein, the term “treatment group” refers to a group of patients or subjects characterized by selection, eligibility, suitability, or otherwise designated for a particular treatment (e.g., medical treatment). Subjects may be assigned to treatment groups based on complement activity biomarker levels that are elevated in comparison to CSF, plasma, and/or serum complement activity biomarker levels in subjects without ALS. Treatment groups may be characterized by eligibility for complement inhibitor treatment. Such complement inhibitor treatment may include zilucoplan treatment. Methods of stratifying subjects with ALS or suspected of having ALS according to the present disclosure may be carried out with subjects with definite or probable ALS, as determined by revised El Escorial criteria.

In some embodiments, the present disclosure provides methods of selecting subjects for complement inhibitor treatment, wherein the subjects have or are suspected of having ALS. Such methods may include assessing the presence or level of biomarkers in subject samples. Such biomarkers may include, but are not limited to, neuroaxonal degeneration biomarkers, neuroinflammation biomarkers, and complement activity biomarkers. Subjects may be selected for complement inhibitor treatment based on the presence or level of biomarkers assessed in subject samples. Subject samples may include plasma and/or CSF samples. Neuroaxonal degeneration biomarkers may include, but are not limited to, pNfH and/or NfL. Neuroinflammation biomarkers may include, but are not limited to, MCP-1, CHIT1, and YKL-40. Complement activity biomarkers may include, but are not limited to, C5a, C5b-9, sC5b-9, C5b6 complex, C3a, C4a, Ba, Bb, Factor I, and Factor H. Complement inhibitors utilized for treatment of selected subject may include zilucoplan. Levels of one or more of Nfl, pNfH, CHIT1, C5a, and sC5b-9 may be elevated in subject samples relative to levels in subject samples from subjects without ALS (non-ALS subjects). Such subject samples may include CSF. Levels of C3a, Bb, and/or Ba may be elevated in CSF samples from subjects with ALS or suspected of having ALS, where subject BBB function is impaired [e.g., as determined by albumin quotient analysis (CSF albumin:plasma serum albumin ratio)]. Such levels may be elevated relative to levels in non-ALS subject CSF samples. Levels of C3a may be elevated in plasma samples from ALS subject with impaired BBB relative to levels in non-ALS subject plasma.

In some embodiments, the present disclosure provides methods of treating subjects with ALS or suspected of having ALS, such methods including selecting a subject for complement inhibitor treatment according to any of the methods described above (e.g., by assessing the presence or level of neuroaxonal degeneration biomarkers, neuroinflammation biomarkers, and/or complement activity biomarkers in a subject sample), and administering a complement inhibitor to the subject. Administered complement inhibitors may include zilucoplan.

In some embodiments, the present disclosure provides compositions that include complement inhibitors for use in methods of treating subjects with ALS or suspected of having ALS, the methods including assessing the presence or level of biomarkers in subject samples and selecting subjects for composition treatment based on the assessment. Biomarkers may include one or more of neuroaxonal degeneration biomarkers, neuroinflammation biomarkers, and complement activity biomarkers. Subject samples may include plasma and/or CSF samples. Neuroaxonal degeneration biomarkers may include pNfH and/or NfL. Neuroinflammation biomarkers may include MCP-1, CHIT1, and/or YKL-40. Complement activity biomarkers may include C5a, C5b-9, sC5b-9, C5b6 complex, C3a, C4a, Ba, Bb, Factor I, and/or Factor H. Biomarker levels may be elevated in ALS subject samples (e.g., CSF samples) relative to levels in samples from subjects without ALS. Subject samples may be obtained from subjects with impaired blood brain barrier function (e.g., as assessed by albumin quotient analysis). C3a, Bb, and/or Ba may be elevated in subject samples (e.g., plasma samples) relative to levels in samples from subjects without ALS. C3a levels may be elevated in subject samples relative to levels in samples from subjects without ALS. Composition complement inhibitors may include C5 inhibitors. C5 inhibitors may include zilucoplan.

Dose and/or regimen of complement inhibitor (e.g., zilucoplan) administered to subjects according to treatment methods described herein may be determined based on levels of complement activity biomarkers detected in CSF, plasma, and/or serum obtained from subjects prior to treatment. Such complement activity biomarkers may be detected in CSF, plasma, and/or serum obtained from subjects prior to treatment may include, but are not limited to, C5a, C5b-9, soluble C5b-9, C3a, C4a, Ba, Bb, Factor I, and Factor H. Dose and/or regimen of complement inhibitor (e.g., zilucoplan) administration may be adjusted after one or more prior administrations based on complement activity biomarker levels detected in CSF, plasma, and/or serum obtained from subjects after one or more prior administration.

In some embodiments, levels of C5a, sC5b-9, and/or Bb in subject CSF are assessed and used for one or more of ALS diagnosis, subject stratification (e.g., into a specific treatment group), ALS monitoring, and determining dose and/or regimen of complement inhibitor (e.g., zilucoplan) treatment. Such assessment may include comparison to levels from healthy subjects (without ALS). In some embodiments, levels of complement activity biomarkers (e.g., C5a, C5b-9, soluble C5b-9, C3a, C4a, Ba, Bb, Factor I, and Factor H) in subject serum may be assessed and used for one or more of ALS diagnosis, subject stratification (e.g., into a specific treatment group), ALS monitoring, and determining dose and/or regimen of complement inhibitor (e.g., zilucoplan) treatment. Such assessment may include comparison to levels from healthy subjects (without ALS).

In some embodiments, ALS treatment methods of the present disclosure include methods of treating ALS by zilucoplan administration to subjects. Zilucoplan inhibits C5a formation in a dose-dependent manner upon activation of the classical pathway and inhibits C5b formation (as measured by C5b-9 or MAC deposition on a complement activating surface) upon activation of the classical and alternative complement pathways. (U.S. Pat. No. 9,937,222). Zilucoplan administration may be subcutaneous (SC) administration. Zilucoplan may be administered at a dose of from about 0.01 mg/kg (mg zilucoplan/kg subject body weight) to about 1.0 mg/kg, from about 0.02 mg/kg to about 2.0 mg/kg, from about 0.05 mg/kg to about 3.0 mg/kg, from about 0.10 mg/kg to about 4.0 mg/kg, from about 0.15 mg/kg to about 4.5 mg/kg, from about 0.20 mg/kg to about 5.0 mg/kg, from about 0.30 mg/kg to about 7.5 mg/kg, from about 0.40 mg/kg to about 10 mg/kg, from about 0.50 mg/kg to about 12.5 mg/kg, from about 0.1 mg/kg to about 0.6 mg/kg, from about 1.0 mg/kg to about 15 mg/kg, from about 2.0 mg/kg to about 20 mg/kg, from about 5.0 mg/kg to about 25 mg/kg, from about 10 mg/kg to about 45 mg/kg, from about 20 mg/kg to about 55 mg/kg, from about 30 mg/kg to about 65 mg/kg, from about 40 mg/kg to about 75 mg/kg, from about 50 mg/kg to about 150 mg/kg, from about 100 mg/kg to about 250 mg/kg, from about 200 mg/kg to about 350 mg/kg, from about 300 mg/kg to about 450 mg/kg, from about 400 mg/kg to about 550 mg/kg, or from about 500 mg/kg to about 1000 mg/kg.

In some embodiments, zilucoplan may be administered at a dose of from about 0.10 mg/kg to about 0.42 mg/kg.

Methods of the present disclosure may include administering zilucoplan at a daily dose of from about 0.1 mg/kg to about 0.3 mg/kg. In some embodiments, zilucoplan is administered at a daily dose of 0.3 mg/kg.

Zilucoplan administration may be by self-administration. Zilucoplan administration may include the use of prefilled syringes. Self-administration may include the use of self-administration devices. Self-administration devices may include or be incorporated with prefilled syringes.

Zilucoplan may be provided in solution. Zilucoplan solutions may include aqueous solutions. Zilucoplan solutions may include phosphate-buffered saline (PBS). Zilucoplan solutions may be preservative-free. Zilucoplan may be present in solutions at a concentration of from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 35 mg/mL to about 55 mg/mL, from about 40 mg/mL to about 60 mg/mL, from about 45 mg/mL to about 75 mg/mL, from about 50 mg/mL to about 100 mg/mL, from about 60 mg/mL to about 200 mg/mL, from about 70 mg/mL to about 300 mg/mL, from about 80 mg/mL to about 400 mg/mL, from about 90 mg/mL to about 500 mg/mL, or from about 100 mg/mL to about 1000 mg/mL.

In some embodiments, self-administration devices include zilucoplan solutions. Self-administration devices may include zilucoplan solution volumes of from about 0.010 mL to about 0.500 mL, from about 0.050 mL to about 0.600 mL, from about 0.100 mL to about 0.700 mL, from about 0.150 mL to about 0.810 mL, from about 0.200 mL to about 0.900 mL, from about 0.250 mL to about 1.00 mL, from about 0.300 mL to about 3.00 mL, from about 0.350 mL to about 3.50 mL, from about 0.400 mL to about 4.00 mL, from about 0.450 mL to about 4.50 mL, from about 0.500 mL to about 5.00 mL, from about 0.550 mL to about 10.0 mL, from about 0.600 mL to about 25.0 mL, from about 0.650 mL to about 50.0 mL, from about 0.700 mL to about 60.0 mL, from about 0.750 mL to about 75.0 mL, from about 0.800 mL to about 80.0 mL, from about 0.850 mL to about 85.0 mL, from about 0.900 mL to about 90.0 mL, from about 0.950 mL to about 95.0 mL, from about 1.00 mL to about 100 mL, from about 2.00 mL to about 200 mL, from about 5.00 mL to about 500 mL, from about 10.0 mL to about 750 mL, from about 25.0 mL to about 800 mL, from about 50.0 mL to about 900 mL, or from about 100 mL to about 1000 mL.

### Formulations

In some embodiments, compounds or compositions, e.g., pharmaceutical compositions, of the present disclosure are formulated in aqueous solutions. In some cases, aqueous solutions further include one or more salt and/or one or more buffering agent. Salts may include sodium chloride which may be included at concentrations of from about 0.05 mM to about 50 mM, from about 1 mM to about 100 mM, from about 20 mM to about 200 mM, or from about 50 mM to about 500 mM. Further solutions may include at least 500 mM sodium chloride. In some cases, aqueous solutions include sodium phosphate. Sodium phosphate may be included in aqueous solutions at a concentration of from about 0.005 mM to about 5 mM, from about 0.01 mM to about 10 mM, from about 0.1 mM to about 50 mM, from about 1 mM to about 100 mM, from about 5 mM to about 150 mM, or from about 10 mM to about 250 mM. In some cases, at least 250 mM sodium phosphate concentrations are used.

Compositions of the present disclosure may include C5 inhibitors at a concentration of from about 0.001 mg/mL to about 0.2 mg/mL, from about 0.01 mg/mL to about 2 mg/mL, from about 0.1 mg/mL to about 10 mg/mL, from about 0.5 mg/mL to about 5 mg/mL, from about 1 mg/mL to about 20 mg/mL, from about 15 mg/mL to about 40 mg/mL, from about 25 mg/mL to about 75 mg/mL, from about 50 mg/mL to about 200 mg/mL, or from about 100 mg/mL to about 400 mg/mL. In some cases, compositions include C5 inhibitors at a concentration of at least 400 mg/mL.

Compositions of the present disclosure may include C5 inhibitors at a concentration of approximately, about or exactly any of the following values: 0.001 mg/mL, 0.2 mg/mL, 0.01 mg/mL, 2 mg/mL, 0.1 mg/mL, 10 mg/mL, 0.5 mg/mL, 5 mg/mL, 1 mg/mL, 20 mg/mL, 15 mg/mL, 40 mg/mL, 25 mg/mL, 75 mg/mL, 50 mg/mL, 200 mg/mL, 100 mg/mL, or 400 mg/mL. In some cases, compositions include C5 inhibitors at a concentration of at least 40 mg/mL.

In some embodiments, compositions of the present disclosure include aqueous compositions including at least water and a C5 inhibitor (e.g., a cyclic C5 inhibitor polypeptide). Aqueous C5 inhibitor compositions may further include one or more salt and/or one or more buffering agent. In some cases, aqueous compositions include water, a cyclic C5 inhibitor polypeptide, a salt, and a buffering agent.

Aqueous C5 inhibitor formulations may have pH levels of from about 2.0 to about 3.0, from about 2.5 to about 3.5, from about 3.0 to about 4.0, from about 3.5 to about 4.5, from about 4.0 to about 5.0, from about 4.5 to about 5.5, from about 5.0 to about 6.0, from about 5.5 to about 6.5, from about 6.0 to about 7.0, from about 6.5 to about 7.5, from about 7.0 to about 8.0, from about 7.5 to about 8.5, from about 8.0 to about 9.0, from about 8.5 to about 9.5, or from about 9.0 to about 10.0.

In some cases, compounds and compositions of the present disclosure are prepared according to good manufacturing practice (GMP) and/or current GMP (cGMP). Guidelines used for implementing GMP and/or cGMP may be obtained from one or more of the US Food and Drug Administration (FDA), the World Health Organization (WHO), and the International Conference on Harmonization (ICH).

### Dosage and Administration

For treatment of human subjects, C5 inhibitors (e.g., zilucoplan and/or active metabolites or variants thereof) may be formulated as pharmaceutical compositions. Depending on the subject to be treated, the mode of administration, and the type of treatment desired (e.g., prevention, prophylaxis, or therapy) C5 inhibitors may be formulated in ways consonant with these parameters. A summary of such techniques is found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

C5 inhibitors (e.g., zilucoplan and/or active metabolites or variants thereof) may be provided in a therapeutically effective amount. In some cases, a therapeutically effective amount of a C5 inhibitor may be achieved by administration of a dose of from about 0.1 mg to about 1 mg, from about 0.5 mg to about 5 mg, from about 1 mg to about 20 mg, from about 5 mg to about 50 mg, from about 10 mg to about 100 mg, from about 20 mg to about 200 mg, or at least 200 mg of one or more C5 inhibitors.

In some embodiments, subjects may be administered a therapeutic amount of a C5 inhibitor (e.g., zilucoplan and/or active metabolites or variants thereof) based on the weight of such subjects. In some cases, C5 inhibitors are administered at a dose of from about 0.001 mg/kg to about 1.0 mg/kg, from about 0.01 mg/kg to about 2.0 mg/kg, from about 0.05 mg/kg to about 5.0 mg/kg, from about 0.03 mg/kg to about 3.0 mg/kg, from about 0.01 mg/kg to about 10 mg/kg, from about 0.1 mg/kg to about 2.0 mg/kg, from about 0.2 mg/kg to about 3.0 mg/kg, from about 0.4 mg/kg to about 4.0 mg/kg, from about 1.0 mg/kg to about 5.0 mg/kg, from about 2.0 mg/kg to about 4.0 mg/kg, from about 1.5 mg/kg to about 7.5 mg/kg, from about 5.0 mg/kg to about 15 mg/kg, from about 7.5 mg/kg to about 12.5 mg/kg, from about 10 mg/kg to about 20 mg/kg, from about 15 mg/kg to about 30 mg/kg, from about 20 mg/kg to about 40 mg/kg, from about 30 mg/kg to about 60 mg/kg, from about 40 mg/kg to about 80 mg/kg, from about 50 mg/kg to about 100 mg/kg, or at least 100 mg/kg. Such ranges may include ranges suitable for administration to human subjects. Dosage levels may be highly dependent on the nature of the condition; drug efficacy; the condition of the patient; the judgment of the practitioner; and the frequency and mode of administration. In some embodiments, zilucoplan and/or active metabolites or variants thereof may be administered at a dose of from about 0.01 mg/kg to about 10 mg/kg. In some cases, zilucoplan and/or active metabolites or variants thereof may be administered at a dose of from about 0.1 mg/kg to about 3 mg/kg.

In some cases, C5 inhibitors (e.g., zilucoplan and/or active metabolites or variants thereof) are provided at concentrations adjusted to achieve a desired level of the C5 inhibitor in a sample, biological system, or subject (e.g., plasma level in a subject). In some cases, desired concentrations of C5 inhibitors in a sample, biological system, or subject may include concentrations of from about 0.001 μM to about 0.01 from about 0.005 μM to about 0.05 μM, from about 0.02 μM to about 0.2 μM, from about 0.03 μM to about 0.3 from about 0.05 μM to about 0.5 from about 0.01 μM to about 2.0 from about 0.1 μM to about 50 from about 0.1 μM to about 10 from about 0.1 μM to about 5 from about 0.2 μM to about 20 from about 5 μM to about 100 or from about 15 μM to about 200 μM. In some cases, desired concentrations of C5 inhibitors in subject plasma may be from about 0.1 μg/mL to about 1000 μg/mL. The desired concentration of C5 inhibitors in subject plasma may be from about 0.01 μg/mL to about 2 μg/mL, from about 0.02 μg/mL to about 4 μg/mL, from about 0.05 μg/mL to about 5 μg/mL, from about 0.1 μg/mL to about 1.0 μg/mL, from about 0.2 μg/mL to about 2.0 μg/mL, from about 0.5 μg/mL to about 5 μg/mL, from about 1 μg/mL to about 5 μg/mL, from about 2 μg/mL to about 10 μg/mL, from about 3 μg/mL to about 9 μg/mL, from about 5 μg/mL to about 20 μg/mL, from about 10 μg/mL to about 40 μg/mL, from about 30 μg/mL to about 60 μg/mL, from about 40 μg/mL to about 80 μg/mL, from about 50 μg/mL to about 100 μg/mL, from about 75 μg/mL to about 150 μg/mL, or at least 150 μg/mL. In other embodiments, C5 inhibitors are administered at a dose sufficient to achieve a maximum serum concentration (Cₘₐₓ) of at least 0.1 μg/mL, at least 0.5 μg/mL, at least 1 μg/mL, at least 5 μg/mL, at least 10 μg/mL, at least 50 μg/mL, at least 100 μg/mL, or at least 1000 μg/mL.

In some embodiments, C5 inhibitors (e.g., zilucoplan and/or active metabolites or variants thereof) are administered daily at a dose sufficient to deliver from about 0.1 mg/day to about 60 mg/day per kg weight of a subject. In some cases, the Cₘₐₓ achieved with each dose is from about 0.1 μg/mL to about 1000 μg/mL. In such cases, the area under the curve (AUC) between doses may be from about 200 μg*hr/mL to about 10,000 μg*hr/mL.

According to some methods of the present disclosure, C5 inhibitors (e.g., zilucoplan and/or active metabolites or variants thereof) are provided at concentrations needed to achieve a desired effect. In some cases, compounds and compositions of the disclosure are provided at an amount necessary to reduce a given reaction or process by half. The concentration needed to achieve such a reduction is referred to herein as the half maximal inhibitory concentration, or “IC₅₀.” Alternatively, compounds and compositions of the disclosure may be provided at an amount necessary to increase a given reaction, activity or process by half. The concentration needed for such an increase is referred to herein as the half maximal effective concentration or “EC₅₀.”

C5 inhibitors (e.g., zilucoplan and/or active metabolites or variants thereof) may be present in amounts totaling 0.1-95% by weight of the total weight of the composition. In some cases, C5 inhibitors are provided by intravenous (IV) administration. In some cases, C5 inhibitors are provided by subcutaneous (SC) administration.

SC administration of C5 inhibitors (e.g., zilucoplan and/or active metabolites or variants thereof) may, in some cases, provide advantages over IV administration. SC administration may allow patients to provide self-treatment. Such treatment may be advantageous in that patients could provide treatment to themselves in their own home, avoiding the need to travel to a provider or medical facility. Further, SC treatment may allow patients to avoid long-term complications associated with IV administration, such as infections, loss of venous access, local thrombosis, and hematomas. In some embodiments, SC treatment may increase patient compliance, patient satisfaction, quality of life, reduce treatment costs and/or drug requirements.

In some cases, daily SC administration provides steady-state C5 inhibitor concentrations that are reached within 1-3 doses, 2-3 doses, 3-5 doses, or 5-10 doses. In some cases, daily SC doses of from about 0.1 mg/kg to about 0.3 mg/kg may achieve sustained C5 inhibitor levels greater than or equal to 2.5 μg/mL and/or inhibition of complement activity of greater than 90%.

C5 inhibitors (e.g., zilucoplan and/or active metabolites or variants thereof) may exhibit slow absorption kinetics (time to maximum observed concentration of greater than 4-8 hours) and high bioavailability (from about 75% to about 100%) after SC administration.

In some embodiments, dosage and/or administration are altered to modulate the half-life (t_{1/2}) of C5 inhibitor levels in a subject or in subject fluids (e.g., plasma). In some cases, t_{1/2} is at least 1 hour, at least 2 hrs, at least 4 hrs, at least 6 hrs, at least 8 hrs, at least 10 hrs, at least 12 hrs, at least 16 hrs, at least 20 hrs, at least 24 hrs, at least 36 hrs, at least 48 hrs, at least 60 hrs, at least 72 hrs, at least 96 hrs, at least 5 days, at least 6 days, at least 7 days, at least 8 days, at least 9 days, at least 10 days, at least 11 days, at least 12 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, or at least 16 weeks.

In some embodiments, C5 inhibitors (e.g., zilucoplan and/or active metabolites or variants thereof) may exhibit long terminal t_{1/2}. Extended terminal t_{1/2} may be due to extensive target binding and/or additional plasma protein binding. In some cases, C5 inhibitors exhibit t_{1/2} values greater than 24 hours in both plasma and whole blood. In some cases, C5 inhibitors do not lose functional activity after incubation in human whole blood at 37° C. for 16 hours.

In some embodiments, dosage and/or administration are altered to modulate the steady state volume of distribution of C5 inhibitors. In some cases, the steady state volume of distribution of C5 inhibitors is from about 0.1 mL/kg to about 1 mL/kg, from about 0.5 mL/kg to about 5 mL/kg, from about 1 mL/kg to about 10 mL/kg, from about 5 mL/kg to about 20 mL/kg, from about 15 mL/kg to about 30 mL/kg, from about 10 mL/kg to about 200 mL/kg, from about 20 mL/kg to about 60 mL/kg, from about 30 mL/kg to about 70 mL/kg, from about 50 mL/kg to about 200 mL/kg, from about 100 mL/kg to about 500 mL/kg, or at least 500 mL/kg. In some cases, the dosage and/or administration of C5 inhibitors is adjusted to ensure that the steady state volume of distribution is equal to at least 50% of total blood volume. In some embodiments, C5 inhibitor distribution may be restricted to the plasma compartment.

In some embodiments, C5 inhibitors (e.g., zilucoplan and/or active metabolites or variants thereof) exhibit a total clearance rate of from about 0.001 mL/hr/kg to about 0.01 mL/hr/kg, from about 0.005 mL/hr/kg to about 0.05 mL/hr/kg, from about 0.01 mL/hr/kg to about 0.1 mL/hr/kg, from about 0.05 mL/hr/kg to about 0.5 mL/hr/kg, from about 0.1 mL/hr/kg to about 1 mL/hr/kg, from about 0.5 mL/hr/kg to about 5 mL/hr/kg, from about 0.04 mL/hr/kg to about 4 mL/hr/kg, from about 1 mL/hr/kg to about 10 mL/hr/kg, from about 5 mL/hr/kg to about 20 mL/hr/kg, from about 15 mL/hr/kg to about 30 mL/hr/kg, or at least 30 mL/hr/kg.

Time periods for which maximum concentration of C5 inhibitors in subjects (e.g., in subject serum) are maintained (Tₘₐₓ values) may be adjusted by altering dosage and/or administration (e.g., subcutaneous administration). In some cases, C5 inhibitors have Tₘₐₓ values of from about 1 min to about 10 min, from about 5 min to about 20 min, from about 15 min to about 45 min, from about 30 min to about 60 min, from about 45 min to about 90 min, from about 1 hour to about 48 hrs, from about 2 hrs to about 10 hrs, from about 5 hrs to about 20 hrs, from about 10 hrs to about 60 hrs, from about 1 day to about 4 days, from about 2 days to about 10 days, or at least 10 days.

In some embodiments, C5 inhibitors (e.g., zilucoplan and/or active metabolites or variants thereof) may be administered without off-target effects. In some cases, C5 inhibitors do not inhibit hERG (human ether-a-go-go related gene), even with concentrations less than or equal to 300 μM. SC injection of C5 inhibitors with dose levels up to 10 mg/kg may be well tolerated and not result in any adverse effects of the cardiovascular system (e.g., elevated risk of prolonged ventricular repolarization) and/or respiratory system.

C5 inhibitor doses may be determined using the no observed adverse effect level (NOAEL) observed in another species. Such species may include, but are not limited to monkeys, rats, rabbits, and mice. In some cases, human equivalent doses (HEDs) may be determined by allometric scaling from NOAELs observed in other species. In some cases, HEDs result in therapeutic margins of from about 2 fold to about 5 fold, from about 4 fold to about 12 fold, from about 5 fold to about 15 fold, from about 10 fold to about 30 fold, or at least 30 fold. In some cases, therapeutic margins are determined by using exposure in primates and estimated human Cₘₐₓ levels in humans.

In some embodiments, C5 inhibitors of the present disclosure allow for a rapid washout period in cases of infection where prolonged inhibition of the complement system is detrimental.

C5 inhibitor administration according to the present disclosure may be modified to reduce potential clinical risks to subjects. Infection with Neisseria meningitidis is a known risk of C5 inhibitors, including eculizumab. In some cases, risk of infection with Neisseria meningitides is minimized by instituting one or more prophylactic steps. Such steps may include the exclusion of subjects who may already be colonized by these bacteria. In some cases, prophylactic steps may include coadministration with one or more antibiotics. In some cases, ciprofloxacin may be coadministered. In some cases, ciprofloxacin may be coadministered orally at a dose of from about 100 mg to about 1000 mg (e.g., 500 mg).

In some embodiments, C5 inhibitors (e.g., zilucoplan and/or active metabolites or variants thereof) are administered at a frequency of every hour, every 2 hrs, every 4 hrs, every 6 hrs, every 12 hrs, every 18 hrs, every 24 hrs, every 36 hrs, every 72 hrs, every 84 hrs, every 96 hrs, every 5 days, every 7 days, every 10 days, every 14 days, every week, every two weeks, every 3 weeks, every 4 weeks, every month, every 2 months, every 3 months, every 4 months, every 5 months, every 6 months, every year, or at least every year. In some cases, C5 inhibitors are administered once daily or administered as two, three, or more sub-doses at appropriate intervals throughout the day.

In some embodiments, C5 inhibitors are administered in multiple daily doses. In some cases, C5 inhibitors are administered daily for 7 days. In some cases, C5 inhibitors are administered daily for 7 to 100 days. In some cases, C5 inhibitors are administered daily for at least 100 days. In some cases, C5 inhibitors are administered daily for an indefinite period.

C5 inhibitors delivered intravenously may be delivered by infusion over a period of time, such as over a 5 minute, 10 minute, 15 minute, 20 minute, or 25 minute period. The administration may be repeated, for example, on a regular basis, such as hourly, daily, weekly, biweekly (i.e., every two weeks), for one month, two months, three months, four months, or more than four months. After an initial treatment regimen, treatments may be administered on a less frequent basis. For example, after biweekly administration for three months, administration may be repeated once per month, for six months or a year or longer. C5 inhibitor administration may reduce, lower, increase or alter binding or any physiologically deleterious process (e.g., in a cell, tissue, blood, urine or other compartment of a patient) by at least 10%, at least 15%, at least 20%, at least 25%, at least 30%, at least 40%, at least 50%, at least 60%, at least 70%, at least 80% or at least 90% or more.

Before administration of a full dose of C5 inhibitor and/or C5 inhibitor composition, patients can be administered a smaller dose, such as 5% of a full dose, and monitored for adverse effects, such as an allergic reaction or infusion reaction, or for elevated lipid levels or blood pressure. In another example, patients can be monitored for unwanted immunostimulatory effects, such as increased cytokine (e.g., TNF-alpha, IL-1, IL-6, or IL-10) levels.

Genetic predisposition plays a role in the development of some diseases or disorders. Therefore, patients in need of C5 inhibitors may be identified by family history analysis, or, for example, screening for one or more genetic markers or variants. Healthcare providers (e.g., doctors or nurses) or family members may analyze family history information before prescribing or administering therapeutic compositions of the present disclosure.

### III. Kits and Devices

In some embodiments, the present disclosure provides kits and devices. Such kits and devices may include any of the compounds or compositions described herein. In a non-limiting example, zilucoplan may be included.

### Kits

In some embodiments, compounds and/or compositions of the present disclosure are provided as part of a kit. Such kits may be used to carry out methods of treating complement-related indications described herein.

Kit components may be packaged in liquid (e.g., aqueous or organic) media or in dry (e.g., lyophilized) form. Kits may include containers that may include, but are not limited to vials, test tubes, flasks, bottles, syringes, or bags. Kit containers may be used to aliquot, store, preserve, insulate, and/or protect kit components. Kit components may be packaged together or separately. Some kits may include containers of sterile, pharmaceutically acceptable buffer and/or other diluent (e.g., phosphate buffered saline). In some embodiments, kits include containers of kit components in dry form with separate containers of solution for dissolving dried components. In some embodiments, kits include a syringe for administering one or more kit components.

When polypeptides are provided as a dried powder it is contemplated that between 10 micrograms and 1000 milligrams of polypeptide, or at least or at most those amounts are provided in kits.

Containers may include at least one vial, test tube, flask, bottle, syringe and/or other receptacle, into which polypeptide formulations may be placed, preferably, suitably allocated. Kits may also include containers for sterile, pharmaceutically acceptable buffer and/or other diluent.

Kits may include instructions for employing kit components as well the use of any other reagent not included in the kit. Instructions may include variations that can be implemented.

In some embodiments, the present disclosure provides kits that include syringes with zilucoplan and instructions. Syringes may be self-injection devices. Self-injection devices may include BD ULTRASAFE PLUS™ self-administration devices (BD, Franklin Lakes, N.J.). Kits may include one or more items for addressing syringe wounds. Such items may include, but are not limited to, alcohol wipes and wound dressings (e.g., cotton balls, mesh pads, bandages, tape, gauze, etc.). Kits may further include disposal containers for disposal of used kit components. Disposal containers may be designed for disposal of sharp objects, such as needles and syringes. Some kits may include instructions for sharp object disposal.

In some embodiments, kits of the present disclosure include zilucoplan in powdered form or in solution. Solutions may be aqueous solutions. Solutions may include PBS. Zilucoplan solutions may include from about 4 mg/ml to about 200 mg/ml zilucoplan. In some embodiments, zilucoplan solutions include about 40 mg/ml zilucoplan. Zilucoplan solutions may include preservatives. In some embodiments, zilucoplan solutions are preservative-free.

### Devices

In some embodiments, compounds and compositions of the present disclosure may be provided as part of a device. Devices may include pre-loaded syringes. As used herein, a “pre-loaded syringe” refers to a delivery device for injection administration, wherein the device is manufactured, prepared, packaged, stored, and/or distributed with a payload to be injected that is included within the device. Due to cyclic peptide stability, cyclic peptide inhibitors are especially well suited for manufacture, storage, and distribution in pre-loaded syringes. Further, pre-loaded syringes are especially well suited for self-administration (i.e., administration by a subject, without the aid of a medical professional). Self-administration represents a convenient way for subjects to obtain treatments without relying on medical professionals who may be located at a distance or are otherwise difficult to access. This makes self-administration options well suited for treatments requiring frequent injections (e.g., daily injections).

In some embodiments, the present disclosure provides pre-loaded syringes for delivery of complement inhibitors. The pre-loaded syringes may include complement inhibitor compositions formulated for injection. The compositions may be formulated for subcutaneous injection. The complement inhibitors may include cyclic peptides. In some embodiments, the pre-loaded syringes include C5 inhibitors. The C5 inhibitors may include zilucoplan or a variant or derivative thereof. Zilucoplan may be included in pre-loaded syringes in a solution of phosphate buffered saline. Zilucoplan may be present in the solution at a concentration of from about 4 mg/ml to about 400 mg/ml. In some embodiments, pre-loaded syringes include a 40 mg/ml solution of zilucoplan in PBS. In some embodiments, the syringes may include a volume of from about 0.1 ml to about 1 ml or from about 0.5 ml to about 2 ml. The solution may include a preservative.

Pre-loaded syringes may include ULTRASAFE PLUS™ passive needle guards (Becton Dickenson, Franklin Lakes, N.J.). Other pre-loaded syringes include injection pens. Injection pens may be multi-dose pens. Some pre-loaded syringes include a needle. In some embodiments, the needle gauge is from about 20 to about 34. The needle gauge may be from about 29 to about 31.

### IV. Definitions

Administered in combination: As used herein, the term “administered in combination” or “combined administration” means that a subject is simultaneously exposed to two or more agents administered at the same time or within an interval of time such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. Bioavailability: As used herein, the term “bioavailability” refers to the systemic availability of a given amount of a compound (e.g., C5 inhibitor) administered to a subject. Bioavailability can be assessed by measuring the area under the curve (AUC) or the maximum serum or plasma concentration (Cₘₐₓ) of the unchanged form of a compound following administration of the compound to a subject. AUC is a determination of the area under the curve when plotting the serum or plasma concentration of a compound along the ordinate (Y-axis) against time along the abscissa (X-axis). Generally, the AUC for a particular compound can be calculated using methods known to those of ordinary skill in the art and/or as described in G. S. Banker, Modern Pharmaceutics, Drugs and the Pharmaceutical Sciences, v. 72, Marcel Dekker, New York, Inc., 1996, the contents of which are herein incorporated by reference in their entirety.

Biological system: As used herein, the term “biological system” refers to a cell, a group of cells, a tissue, an organ, a group of organs, an organelle, a biological fluid, a biological signaling pathway (e.g., a receptor-activated signaling pathway, a charge-activated signaling pathway, a metabolic pathway, a cellular signaling pathway, etc.), a group of proteins, a group of nucleic acids, or a group of molecules (including, but not limited to biomolecules) that carry out at least one biological function or biological task within cellular membranes, cellular compartments, cells, cell cultures, tissues, organs, organ systems, organisms, multicellular organisms, biological fluids, or any biological entities. In some embodiments, biological systems are cell signaling pathways that include intracellular and/or extracellular signaling biomolecules. In some embodiments, biological systems include proteolytic cascades (e.g., the complement cascade).

Buffering agent: As used herein, the term “buffering agent” refers to a compound used in a solution for the purposes of resisting changes in pH. Such compounds may include, but are not limited to acetic acid, adipic acid, sodium acetate, benzoic acid, citric acid, sodium benzoate, maleic acid, sodium phosphate, tartaric acid, lactic acid, potassium metaphosphate, glycine, sodium bicarbonate, potassium phosphate, sodium citrate, and sodium tartrate.

Clearance rate: As used herein, the term “clearance rate” refers to the velocity at which a particular compound is cleared from a biological system or fluid.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art will appreciate that some compounds exist in different forms, show different properties and/or activities (including, but not limited to biological activities). In such cases it is within the ordinary skill of those in the art to select or avoid particular forms of a compound for use in accordance with the present disclosure. For example, compounds that contain asymmetrically substituted carbon atoms can be isolated in optically active or racemic forms.

Cyclic or Cyclized: As used herein, the term “cyclic” refers to the presence of a continuous loop. The continuous loop may be formed by a chemical bond between different regions of a compound (also referred to herein as a “cyclic bond.”) Cyclic molecules need not be circular, only joined to form an unbroken chain of subunits. Cyclic polypeptides may include a “cyclic loop,” formed when two amino acids are connected by a bridging moiety. The cyclic loop comprises the amino acids along the polypeptide present between the bridged amino acids. Cyclic loops may include 2, 3, 4, 5, 6, 7, 8, 9, 10 or more amino acids.

Downstream event: As used herein, the term “downstream” or “downstream event,” refers to any event occurring after and/or as a result of another event. In some cases, downstream events are events occurring after and as a result of C5 cleavage and/or complement activation. Such events may include, but are not limited to, generation of C5 cleavage products, activation of MAC, hemolysis, and hemolysis-related disease (e.g., PNH).

Equilibrium dissociation constant: As used herein, the term “equilibrium dissociation constant” or “K_{D}” refers to a value representing the tendency of two or more agents (e.g., two proteins) to reversibly separate. In some cases, K_{D} indicates a concentration of a primary agent at which half of the total levels of a secondary agent are associated with the primary agent.

Half-life: As used herein, the term “half-life” or “t_{1/2}” refers to the time it takes for a given process or compound concentration to reach half of a final value. The “terminal half-life” or “terminal t_{1/2}” refers to the time needed for the plasma concentration of a factor to be reduced by half after the concentration of the factor has reached a pseudo-equilibrium.

Identity: As used herein, the term “identity,” when referring to polypeptides or nucleic acids, refers to a comparative relationship between sequences. The term is used to describe the degree of sequence relatedness between polymeric sequences and may include the percentage of matching monomeric components with gap alignments (if any) addressed by a particular mathematical model or computer program (i.e., “algorithms”). Identity of related polypeptides can be readily calculated by known methods. Such methods include, but are not limited to, those described previously by others (Lesk, A. M., ed., Computational Molecular Biology, Oxford University Press, New York, 1988; Smith, D. W., ed., Biocomputing: Informatics and Genome Projects, Academic Press, New York, 1993; Griffin, A. M. et al., ed., Computer Analysis of Sequence Data, Part 1, Humana Press, New Jersey, 1994; von Heinje, G., Sequence Analysis in Molecular Biology, Academic Press, 1987; Gribskov, M. et al., ed., Sequence Analysis Primer, M. Stockton Press, New York, 1991; and Carillo et al., Applied Math, SIAM J, 1988, 48, 1073).

Inhibitor: As used herein, the term “inhibitor” refers to any agent that blocks or causes a reduction in the occurrence of a specific event; cellular signal; chemical pathway; enzymatic reaction; cellular process; interaction between two or more entities; biological event; disease; disorder; or condition.

Initial loading dose: As used herein, an “initial loading dose” refers to a first dose of a therapeutic agent that may differ from one or more subsequent doses. Initial loading doses may be used to achieve an initial concentration of a therapeutic agent or level of activity before subsequent doses are administered.

Intravenous: As used herein, the term “intravenous” refers to the area within a blood vessel. Intravenous administration typically refers to delivery of a compound into the blood through injection in a blood vessel (e.g., vein).

In vitro: As used herein, the term “in vitro” refers to events that occur in an artificial environment (e.g., in a test tube or reaction vessel, in cell culture, in a Petri dish, etc.), rather than within an organism (e.g., animal, plant, or microbe).

In vivo: As used herein, the term “in vivo” refers to events that occur within an organism (e.g., animal, plant, or microbe or cell or tissue thereof).

Lactam bridge: As used herein, the term “lactam bridge” refers to an amide bond that forms a bridge between chemical groups in a molecule. In some cases, lactam bridges are formed between amino acids in a polypeptide.

Linker: The term “linker” as used herein refers to a group of atoms (e.g., 10-1,000 atoms), molecule(s), or other compounds used to join two or more entities. Linkers may join such entities through covalent or non-covalent (e.g., ionic or hydrophobic) interactions. Linkers may include chains of two or more polyethylene glycol (PEG) units. In some cases, linkers may be cleavable.

Minute volume: As used herein, the term “minute volume” refers to the volume of air inhaled or exhaled from a subject's lungs per minute.

Non-proteinogenic: As used herein, the term “non-proteinogenic” refers to any non-natural proteins, such as those with non-natural components, such as non-natural amino acids.

Patient: As used herein, “patient” refers to a subject who may seek or be in need of treatment, requires treatment, is receiving treatment, will receive treatment, or a subject who is under the care of a trained professional for a particular disease or condition.

Pharmaceutical composition: As used herein, the term “pharmaceutical composition” refers to a composition with at least one active ingredient (e.g., a C5 inhibitor) in a form and amount that permits the active ingredient to be therapeutically effective.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers to any ingredient other than active agents (e.g., active agent zilucoplan and/or active metabolites thereof or variants thereof) present in a pharmaceutical composition and having the properties of being substantially nontoxic and non-inflammatory in a patient. In some embodiments, a pharmaceutically acceptable excipient is a vehicle capable of suspending or dissolving the active agent. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Plasma compartment: As used herein, the term “plasma compartment” refers to intravascular space occupied by blood plasma.

Salt: As used herein, the term “salt” refers to a compound made up of a cation with a bound anion. Such compounds may include sodium chloride (NaCl) or other classes of salts including, but not limited to acetates, chlorides, carbonates, cyanides, nitrites, nitrates, sulfates, and phosphates. The term “salt” may also be used to refer to salt forms of polypeptides described herein (e.g., zilucoplan salt). Such polypeptide salts may include zilucoplan sodium salt.

Sample: As used herein, the term “sample” refers to an aliquot or portion taken from a source and/or provided for analysis or processing. In some embodiments, a sample is from a biological source (referred to herein as a “biological sample”) such as a tissue, cell or component part (e.g., a body fluid, including but not limited to blood, mucus, lymphatic fluid, synovial fluid, cerebrospinal fluid, spinal fluid, saliva, amniotic fluid, amniotic cord blood, urine, vaginal fluid and semen). In some embodiments, a sample may be or include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, or organs. In some embodiments, a sample is or includes a medium, such as a nutrient broth or gel, which may contain cellular components, such as proteins. In some embodiments, a “primary” sample is an aliquot of the source. In some embodiments, a primary sample is subjected to one or more processing (e.g., separation, purification, etc.) steps to prepare a sample for analysis or other use.

Subcutaneous: As used herein, the term “subcutaneous” refers to the space underneath the skin. Subcutaneous administration is delivery of a compound beneath the skin.

Subject: As used herein, the term “subject” refers to any organism to which a compound or method in accordance with the disclosure may be administered or applied, e.g., for experimental, diagnostic, prophylactic, and/or therapeutic purposes. Typical subjects include animals (e.g., mammals such as mice, rats, rabbits, porcine subjects, non-human primates, and humans).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” means an amount of an agent to be delivered (e.g., C5 inhibitor) that is sufficient, when administered to a subject suffering from or susceptible to a disease, disorder, and/or condition, to treat, improve symptoms of, diagnose, prevent, and/or delay the onset of the disease, disorder, and/or condition.

Tidal volume: As used herein, the term “tidal volume” refers to the normal lung volume of air displaced between breaths (in the absence of any extra effort).

Tₘₐₓ: As used herein, the term “Tₘₐₓ” refers to the time period for which maximum concentration of a compound in a subject or fluid is maintained.

Treating: As used herein, the term “treating” refers to partially or completely alleviating, ameliorating, improving, relieving, delaying onset of, inhibiting progression of, reducing severity of, and/or reducing incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Treatment dose: As used herein, “treatment dose” refers to one or more doses of a therapeutic agent administered in the course of addressing or alleviating a therapeutic indication. Treatment doses may be adjusted to maintain a desired concentration or level of activity of a therapeutic agent in a body fluid or biological system.

Volume of distribution: As used herein, the term “volume of distribution” or “V_{dist}” refers to a fluid volume required to contain the total amount of a compound in the body at the same concentration as in the blood or plasma. The volume of distribution may reflect the extent to which a compound is present in the extravascular tissue. A large volume of distribution reflects the tendency of a compound to bind to tissue components compared with plasma protein components. In a clinical setting, V_{dist} can be used to determine a loading dose of a compound to achieve a steady state concentration of that compound.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Preparation of Zilucoplan Aqueous Solution

Polypeptides were synthesized using standard solid-phase Fmoc/tBu methods. The synthesis was performed on a Liberty automated microwave peptide synthesizer (CEM, Matthews NC) using standard protocols with Rink amide resin, although other automated synthesizers without microwave capability may also be used. All amino acids were obtained from commercial sources. The coupling reagent used was 2-(6-chloro-1-H-benzotriazole-1yl)-1,1,3,3-tetramethylaminium hexafluorophosphate (HCTU) and the base was diisopropylethylamine (DIEA). Polypeptides were cleaved from resin with 95% TFA, 2.5% TIS and 2.5% water for 3 hours and isolated by precipitation with ether. The crude polypeptides were purified on a reverse phase preparative HPLC using a C18 column, with an acetonitrile/water 0.1% TFA gradient from 20%-50% over 30 min. Fractions containing pure polypeptides were collected and lyophilized and all polypeptides were analyzed by LC-MS.

Zilucoplan (SEQ ID NO: 1), as described in International Publication Numbers WO2017105939 and WO2018106859 (see also CAS Number 1841136-73-9), was prepared as a cyclic peptide containing 15 amino acids (4 of which are non-natural amino acids), an acetylated N-terminus, and a C-terminal carboxylic acid. The C-terminal lysine of the core peptide has a modified side chain, forming a N-ε-(PEG24-γ-glutamic acid-N-α-hexadecanoyl) lysine reside. This modified side chain includes a polyethyleneglycol spacer (PEG24) attached to an L-γ glutamic acid residue that is derivatized with a palmitoyl group. The cyclization of zilucoplan is via a lactam bridge between the side-chains of L-Lys1 and L-Asp6. All of the amino acids in zilucoplan are L-amino acids. Zilucoplan has a molecular weight of 3562.23 g/mol and a chemical formula of C₁₇₂H₂₇₈N₂₄O₅₅.

Like eculizumab, zilucoplan blocks the proteolytic cleavage of C5 into C5a and C5b. Unlike eculizumab, zilucoplan can also bind to C5b and block C6 binding which prevents the subsequent assembly of the MAC.

Zilucoplan was prepared as an aqueous solution for injection containing 40 mg/mL of zilucoplan in a formulation of 50 mM sodium phosphate and 75.7 mM sodium chloride at a pH of 7.0. The resulting composition was used to prepare a medicinal product, in accordance with current Good Manufacturing Practices (cGMPs), the medicinal product including a 1 ml glass syringe with a 29 gauge, ½ inch staked needle placed within a needle safety self-administration device (ULTRASAFE PLUS™, Becton Dickenson, Franklin Lakes, N.J.) with finger flange and plunger rod.

### Example 2. Zilucoplan Administration and Storage

Zilucoplan is administered by subcutaneous (SC) or intravenous (IV) injection and the dose administered (dose volume) is adjusted based on subject weight on a mg/kg basis. This is achieved using a set of fixed doses aligned to a set of weight brackets. In total, human dosing supports a broad weight range of 43 to 109 kg (see Table 2). Injection volumes are approximately 0.4 mL to 0.8 mL. Subjects who present with a higher body weight (>109 kg) are accommodated on a case-by-case basis, in consultation with a medical monitor.

TABLE 2
Dosage presentation by weight group

| Target | | | Weight | Dose |
|---|---|---|---|---|
| dose | Dose presentation | | range | range |
| (mg/kg) | Fill volume (mL) | Dose (mg) | (kg) | (mg/kg) |
| 0.3 | 0.416 | 16.6 | ≥43 to <56 | 0.30 to 0.39 |
| 0.3 | 0.574 | 23 | ≥56 to <77 | 0.30 to 0.41 |
| 0.3 | 0.810 | 32.4 | ≥77 to <109 | 0.30 to 0.42 |

Zilucoplan is stored at 2° C. to 8° C. [36° F. to 46° F.]. Once dispensed to subjects, zilucoplan is stored at controlled room temperature (20° C. to 25° C. [68° F. to 77° F.]) for up to 30 days and is protected from sources of excessive temperature fluctuations such as high heat or exposure to light. Storage of zilucoplan outside of room temperatures is preferably avoided. Zilucoplan may be stored for up to 30 days under these conditions.

### Example 3. Zilucoplan Treatment of ALS

ALS treatment with zilucoplan is carried out as part of a randomized, double-blind, and placebo-controlled study, followed by an open label, long-term extension. Study participants are selected from a broad patient population of individuals diagnosed with ALS that includes individuals from both familial and sporadic ALS patient populations with ALS diagnosed as possible, probable, lab-supported probable, or definite ALS (according to revised El Escorial criteria, see Ludolph, A. et al., ALS and FD, 2015. Early Online: 1-2), with less than or equal to three years since weakness onset, greater than fifty percent predicted vital capacity (based on sex, age, and height), and absent or stable dosing of riluzole or edaravone (at least one or more treatment cycle) for greater than or equal to 30 days at the time of screening.

Zilucoplan and matching placebo are supplied as sterile, preservative-free, aqueous solutions prefilled into 1 mL glass syringes (BD ULTRASAFE PLUS™ self-administration device, BD, Franklin Lakes, N.J.) with 29 gauge, ½ inch, staked needles placed within self-administration devices. Fill volumes are adjusted based on subject weight range to achieve correct mg/kg dose range. Subjects are instructed to self-administer SC doses daily.

During the Treatment Period, subjects return to the clinic at predetermined intervals to evaluate safety, tolerability, and efficacy. Primary endpoint of the study is change in ALS Functional Rating Scale-Revised (ALSFRS-R) score from baseline through 24 weeks of treatment. Plasma and CSF concentrations of C5, zilucoplan, and any zilucoplan metabolites are assessed from samples taken during the study. Samples are also tested for complement activity by hemolysis assays and assays for detection of C5 cleavage products.

Treatment efficacy is also determined using various assessments carried out during clinical visits. These include slow vital capacity (SVC) assessment, muscle strength assessment by hand held dynamometry (HHD), speech capability assessment (including assessment of one or more of articulatory precision, speaking rate, percent of speech signal voiced, phonation length, consonant length, and nasality), and ALS biomarker profile assessment [including assessment of levels of one or more of neurofilament heavy chain (pNfH), neurofilament light chain (NfL), neurotrophin receptor p75 extracellular domain (p75ECD), soluble C5b-9, and C5 cleavage products]. Some efficacy determinations include home-based self-assessments that may be carried out with the use of a smart device (watch, mobile phone, computer, etc.).

Other laboratory assessments are also carried out during clinical visits. These include hematology panel assessments (hematocrit, hemoglobin, platelet count, red blood cells indices, total red blood cells, total white blood cells, and white blood cell & differential); blood chemistry panel/liver function tests (alanine aminotransferase, aspartate aminotransferase, albumin, alkaline phosphatase, bicarbonate, blood urea nitrogen, calcium, chloride, creatinine, glucose, magnesium, phosphate, potassium, sodium, total bilirubin and total protein, and uric acid); and urinalysis (albumin, bilirubin, blood, clarity, color, glucose, ketones, nitrate, pH, protein, specific gravity, urobilinogen, and white blood cell screen). Standard 12-lead ECG is also performed for review of tracings obtained. Physical and neurological examinations are performed to examine head/neck, eyes, ears, nose/throat, cardiovascular system, lungs, abdomen, musculoskeletal system, central nervous system, extremities, and skin.

Subjects treated with zilucoplan exhibit reduced or stabilized disease severity.

### Example 4. Direct Assessment of Complement C5 Activity in CSF

Cerebrospinal fluid (CSF) samples obtained from human subjects treated with or without zilucoplan are assayed for complement activity and related biomarkers. C5 activity levels in CSF samples are assessed by combining CSF samples with C5-depleted normal human serum and testing for ability to promote red blood cell lysis (based on CSF-derived C5). CSF samples are also analyzed for zilucoplan levels, C5 levels, and levels of C5 activity biomarkers [e.g., soluble C5b-9 (sC5b-9)]. Samples from zilucoplan-treated subjects exhibited lower hemolysis activity levels and lower levels of C5 activity biomarkers with levels correlating with treatment-dependent zilucoplan inhibitor levels.

### Example 5. Central Nervous System Complement Synthesis

C5-deficient mice were transplanted with human liver to enable expression of human C5. At a subsequent time point, mice were euthanized and serum and brain tissue samples were analyzed for human C5 levels. Human C5 was detected in mouse serum, but not in brain tissue. These results indicate that C5 does not cross the blood brain barrier (BBB) and that expression in brain is due to local synthesis.

### Example 6. Complement Activity Biomarker Analysis

CSF and matching serum samples from human subjects with ALS (n=20) or without ALS (n=14) were assessed for levels of C5 and complement activity biomarkers. C5 levels were assessed by enzyme-linked immunosorbent assay (ELISA) using a commercial kit (catalog number ab125963; Abcam, Cambridge, UK) according to manufacturer instructions. Complement activity biomarkers were assessed by Quidel Complement Multiplex LLOQ assay (Quidel Corporation, San Diego, Calif.). Results are presented in Table 3. In the Table, p-values represent level of significance over corresponding healthy control levels (as determined by Mann-Whitney test).

TABLE 3
Biomarker levels

| | Healthy | | | Healthy | | |
|---|---|---|---|---|---|---|
| | Control | ALS | | Control | ALS | |
| | CSF | CSF | Significance | Serum | Serum | Significance |
| Biomarkers | (ng/mL) | (ng/mL) | (P-value) | (ng/mL) | (ng/mL) | (P-value) |
| C5 | 153.60 | 142.75 | 0.7282 | 86140 | 88700 | 0.5327 |
| C5a | 0.04 | 0.41 | 0.0087 | 10.46 | 23.06 | 0.0001 |
| sC5b-9 | 3.98 | 25.95 | 0.0006 | 340.1 | 2393 | <0.0001 |
| C3a | 2.49 | 4.15 | 0.0650 | 787.0 | 1470 | 0.0081 |
| C4a | 25.40 | 25.42 | 0.9935 | 410.7 | 1187 | 0.0003 |
| Ba | 7.69 | 12.48 | 0.1232 | 760.9 | 1065 | 0.0007 |
| Bb | 4.64 | 9.77 | 0.0283 | 1374 | 1843 | 0.0105 |
| Factor I (fI) | 177.10 | 226.60 | 0.2075 | 259700 | 197100 | 0.006 |
| Factor H (fH) | 2036.00 | 2065.00 | 0.8575 | 234100 | 219900 | 0.4069 |

Levels of complement activity biomarkers C5a, sC5b-9, and Bb were significantly elevated in ALS CSF samples in comparison to samples from healthy controls. Levels of all complement activity biomarkers tested were significantly elevated in serum samples from subjects with ALS. Interestingly, no statistically significant differences in C5 CSF or serum levels were observed between healthy subjects and subjects with ALS, indicating that C5 is being replenished in CSF and serum compartments upon consumption. CSF levels of C5 were about 600 times lower than serum levels. Overall, these results indicate that complement activity biomarkers in subject serum and C5a, sC5b-9, and Bb levels in subject CSF correlate with ALS.

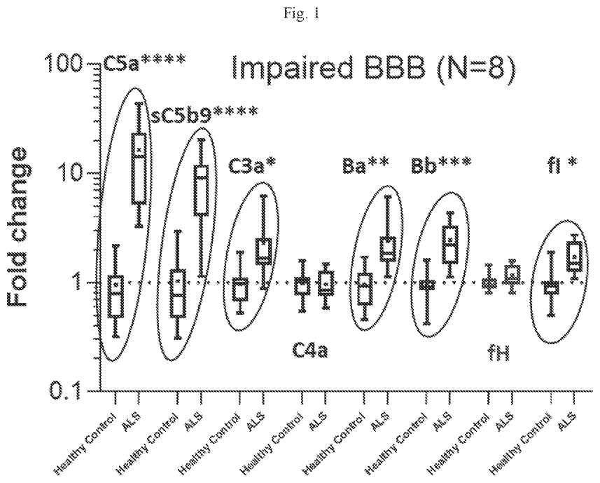

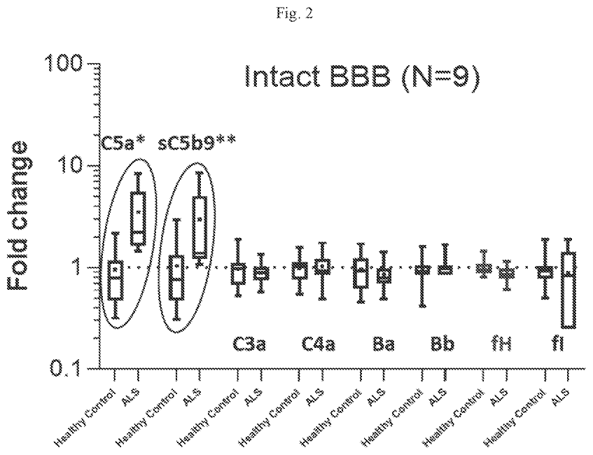

The ratio of CSF albumin levels to plasma albumin levels for each subject was calculated to obtain albumin-quotient values as a measure of BBB permeability. Albumin-quotient values indicated BBB impairment (greater than 9.0) for 47% of subjects with ALS and only 7% for healthy subjects. CSF biomarker levels were compared between subjects with impaired BBB (FIG. 1) and intact BBB (FIG. 2) based on albumin-quotient determination to evaluate complement activity and predominant complement activation pathway (classical, alternative, or lectin pathway). Markers of the alternative activation pathway (C3a, Ba and Bb) were significantly elevated in CSF of subjects with impaired BBB (FIG. 1). Interestingly, only markers of classical or lectin activation pathway, but not alternative pathway, were observed with similar biomarker analysis in corresponding plasma.

### Example 7. Subpopulation Analysis

Complement activity biomarker analysis was conducted with CSF and matching plasma samples from human subjects with probable ALS and results were compared with those from subjects with definite ALS and healthy controls (as described in Example 6, above). Subjects with “definite” or “probable” ALS were identified according to revised El Escorial criteria as described in Brooks R B, et al. Amyotroph Lateral Scler Other Motor Neuron Disord. 2000 December; 1(5):293-9. Complete details related to subjects from which samples were obtained and analyzed are presented in Table 4.

TABLE 4
Population characteristics

| | Subjects with definite or | | | |
|---|---|---|---|---|
| | probable ALS: | | Healthy control | |
| Characteristic | CSF: n = 28, plasma: n = 25 | | subjects: n = 14 | |
| Gender: female/male (ratio) | 8/20 | (0.4) | 7/7 | (1) |
| Age at biospecimen sampling: | 65 | (41-80) | 65 | (54-70) |
| median (range) | | | | |
| Symptom duration in months at sampling: | 15.5 | (2-56) | | |
| median (range) | | | | |
| Inheritance: familial/sporadic (ratio) | 2/26 | (0.077) | | |
| Clinical presentation (revised El Escorial | | | | |
| criteria): | | | | |
| Definite | 21 | | | |
| Probable | 7 | | | |
| ALSFRS-R score | | | | |

| | |
|---|---|
| Score 40-48, mild to minimal | 18 |
| Score 30-39, moderate to mild | 9 |
| Score 21-29, severe to moderate | 1 |

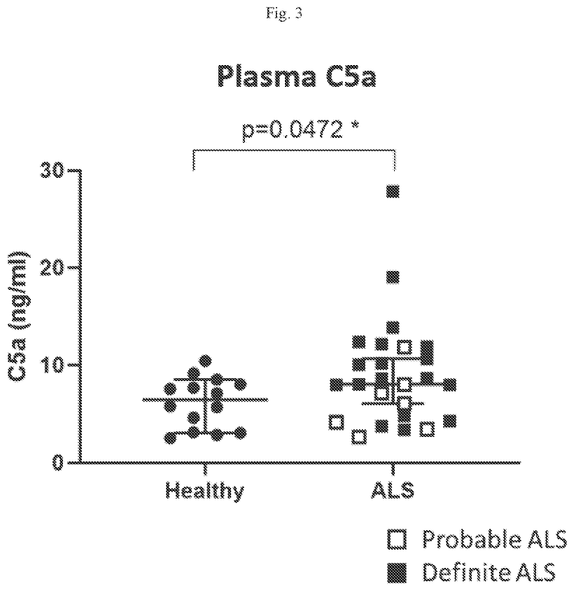

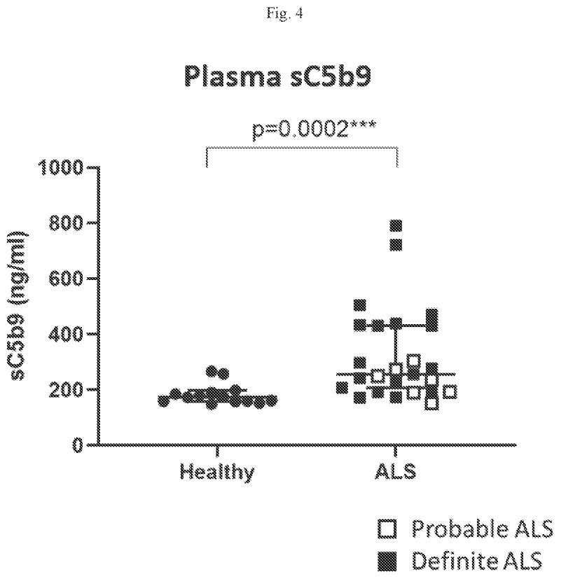

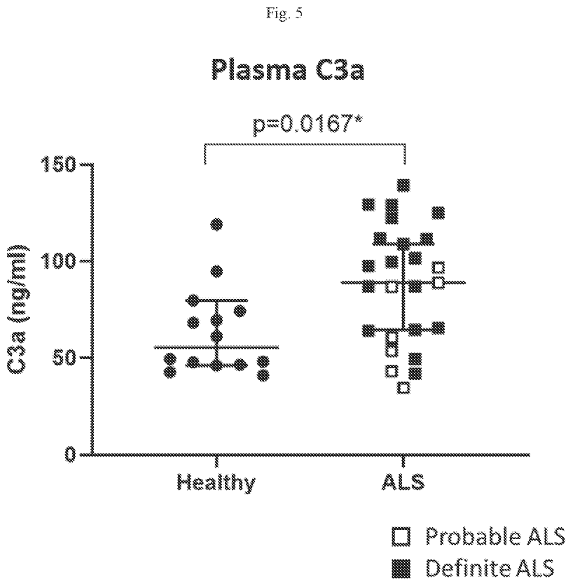

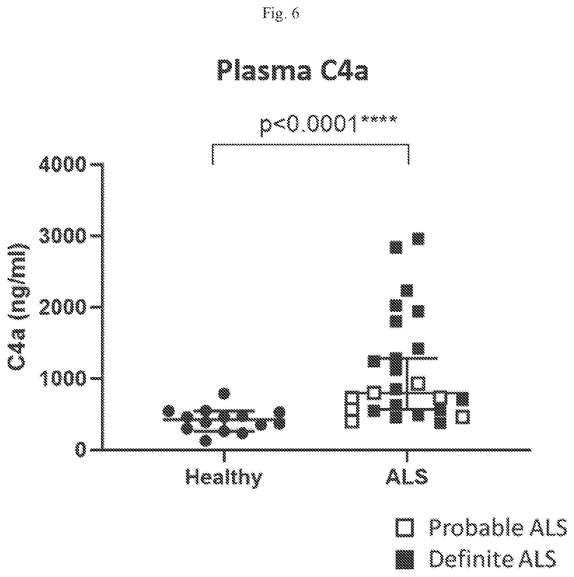

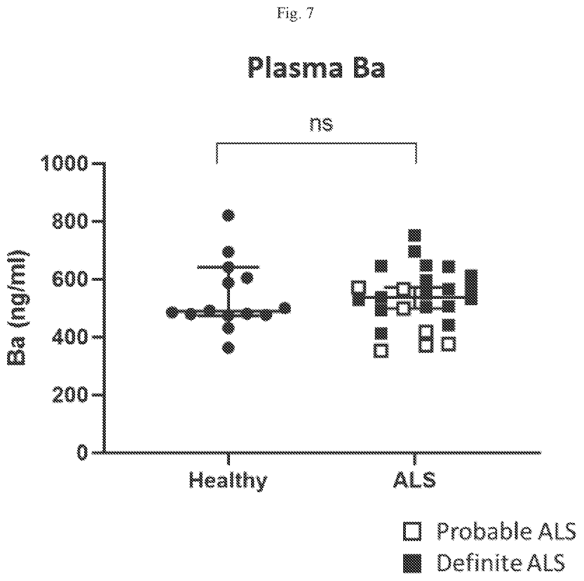

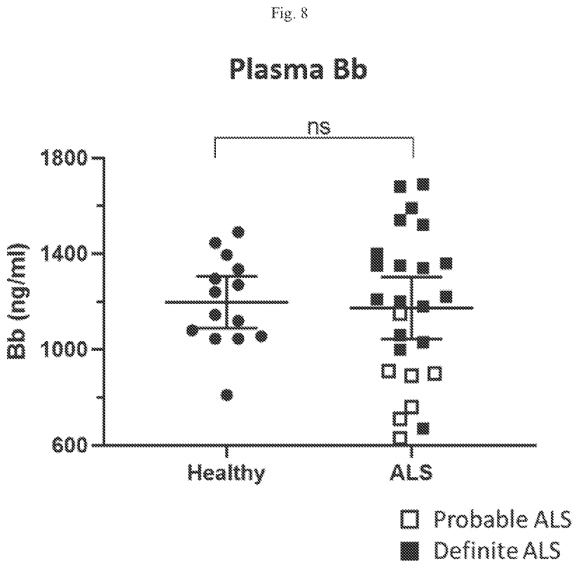

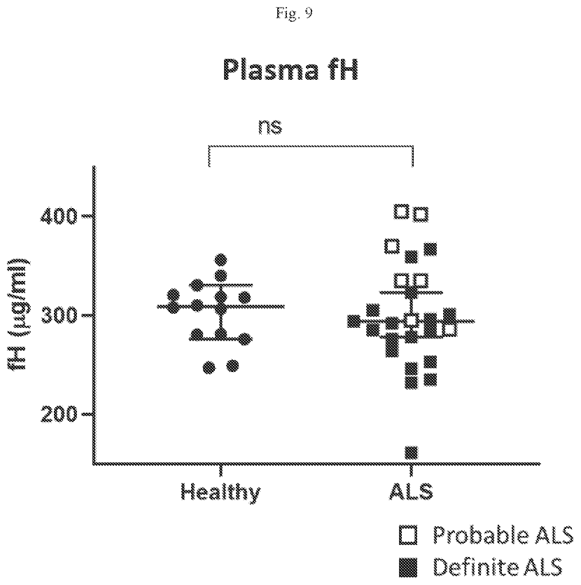

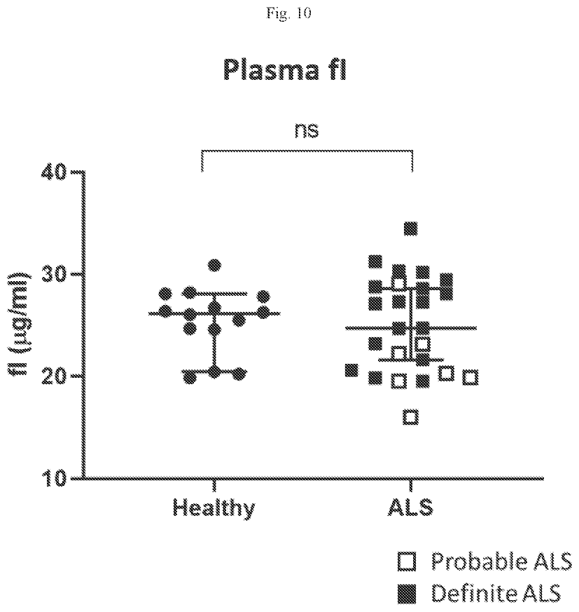

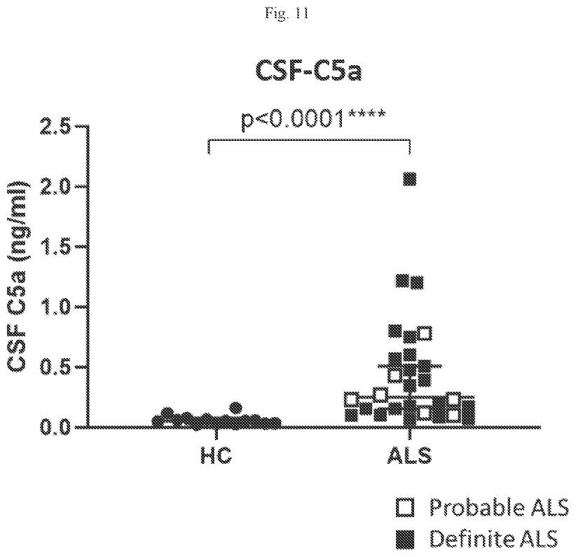

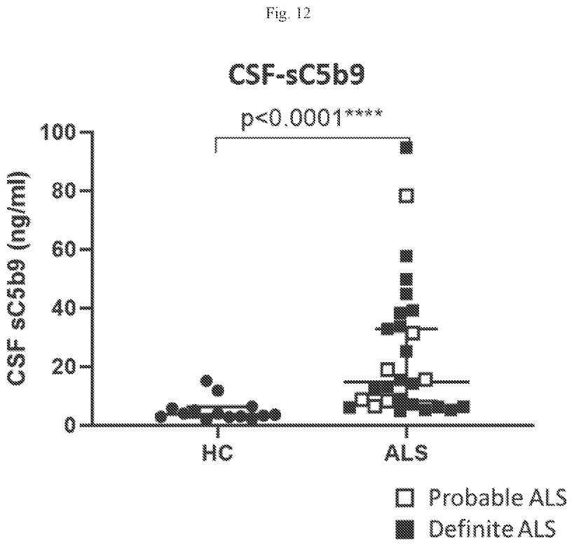

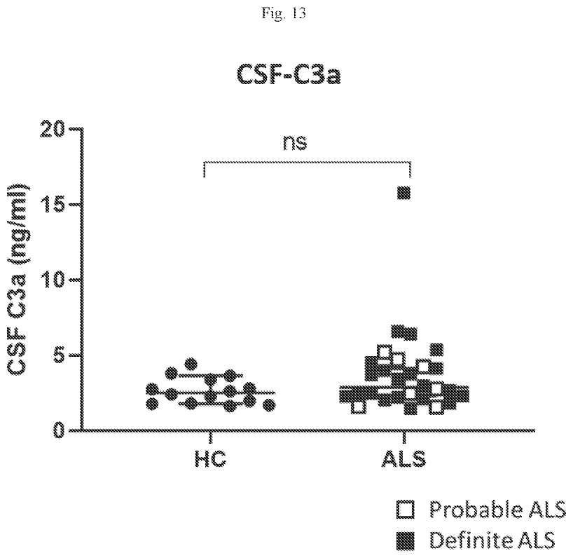

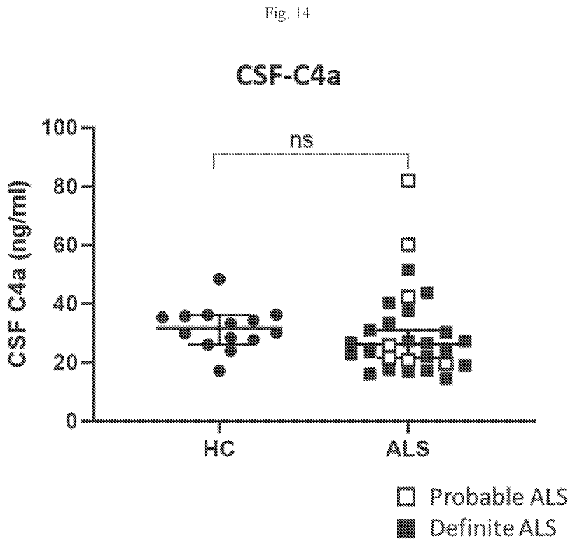

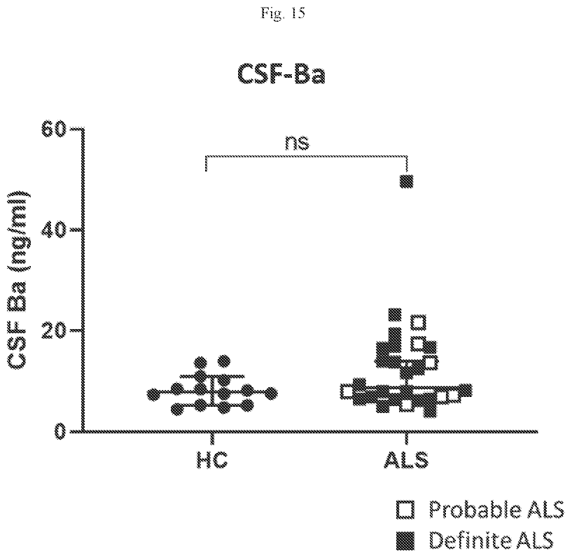

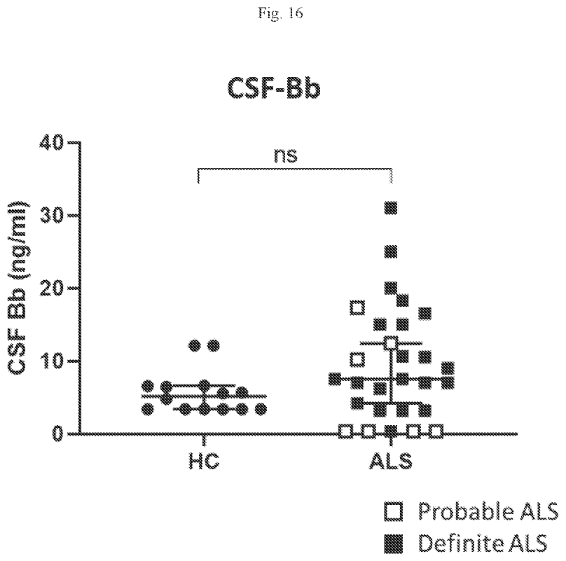

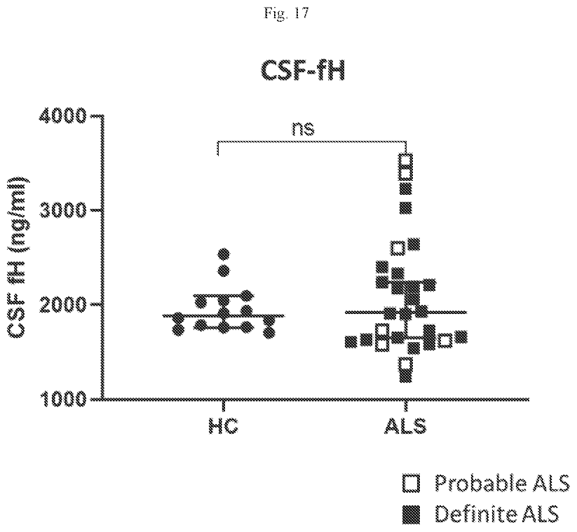

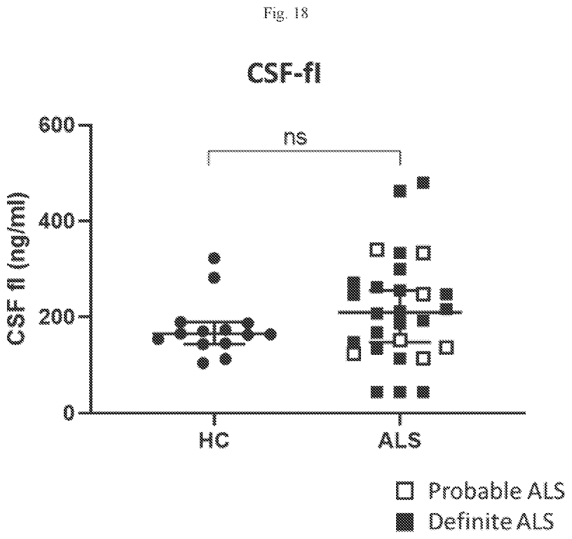

In plasma samples analyzed, C5a (FIG. 3), sC5b9 (FIG. 4), C3a (FIG. 5), and C4a (FIG. 6) levels were significantly elevated in subjects with ALS (definite and probable combined) as compared to healthy controls. Differences in Ba (FIG. 7), Bb (FIG. 8), fH (FIG. 9), and fI (FIG. 10) biomarker levels were not significant between healthy control plasma and plasma from subjects ASL (definite and probable ALS combined). In CSF, only C5a (FIG. 11) and sC5b9 (FIG. 12) levels were significantly elevated with definite or probable ALS as compared to CSF from healthy controls, while C3a (FIG. 13), C4a (FIG. 14), Ba (FIG. 15), Bb (FIG. 16), fH (FIG. 17), and fI (FIG. 18) biomarker levels were not.

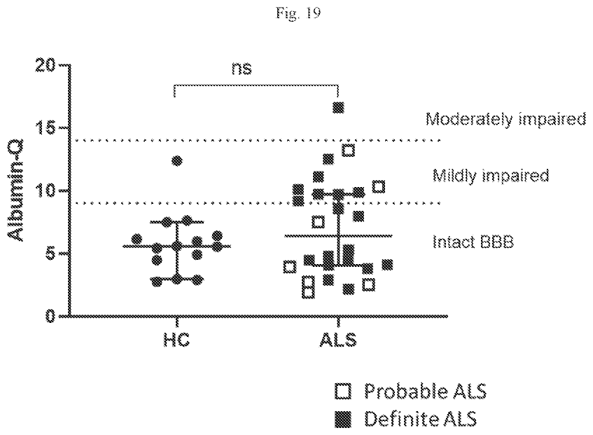

Albumin quotient values were also compared between subjects with definite or probable ALS and healthy control subjects (see FIG. 19). 36% of subjects with definite or probable ALS were determined to have blood brain barrier impairment compared to only 7% of healthy controls.

Overall, C5a and sC5b-9 levels produced in the CSF did not correlate with those observed in plasma. This finding, when taken together with observed elevation in complement split products in CSF in the absence of BBB impairment, supports local CNS complement activity in ALS that is independent from activity in the periphery.

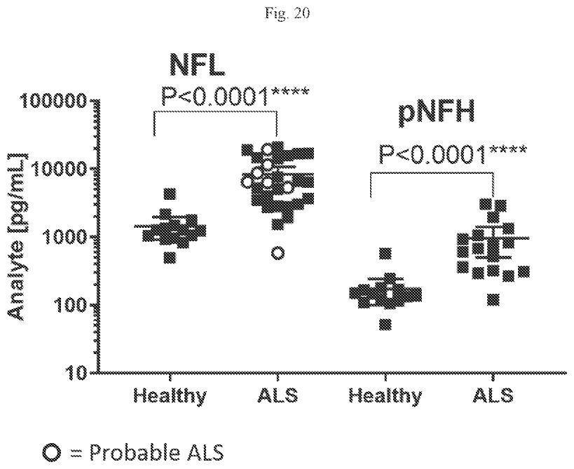

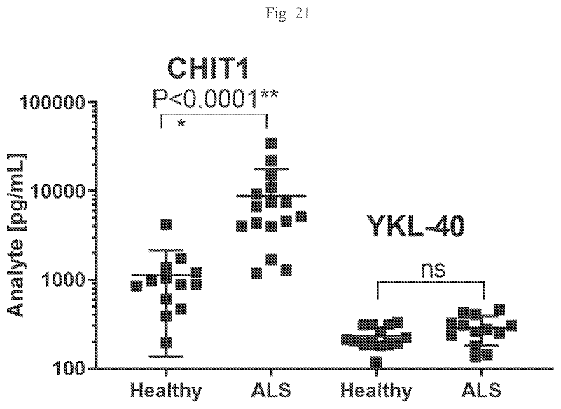

Levels of biomarkers associated with neuroaxonal degeneration (NfL and pNfH) and neuroinflammation [chitotriosidase-1 (CHIT1) and chitinase-3-like protein 1 (YKL-40)] were also analyzed in CSF samples from healthy control subjects and subjects with probable or definite ALS. Both NfL and pNfH levels were elevated in CSF from subjects with probable or definite ALS as compared to healthy control (FIG. 20). Levels of CHIT1 were also elevated in CSF samples from subjects with ALS as compared to healthy controls, while no statistical difference was observed in YKL-40 levels (FIG. 21).

### Example 8. Expanded Subpopulation Analysis

Complement activity biomarker analysis described in the previous Example was repeated with additional CSF and matching plasma samples from healthy human control subjects, human subjects with probable ALS, or human subjects with definite ALS. Subjects with “definite” or “probable” ALS were identified according to revised El Escorial criteria as described in Brooks R B, et al. Amyotroph Lateral Scler Other Motor Neuron Disord. 2000 December; 1(5):293-9. In the expanded analysis, subjects with “probable” ALS included: (1) subjects diagnosed based on presence of upper motor neuron (UMN) and lower motor neuron (LMN) signs in at least two regions with UMN signs rostral to LMN signs; and (2) subjects diagnosed with laboratory results-supported probable ALS based on presence of UMN and LMN signs in one region with evidence by electromyography (EMG) of LMN involvement in another region. Complete details related to subjects from which samples were obtained and analyzed are presented in Table 5.

TABLE 5

| | Subjects with definite or | | | |
|---|---|---|---|---|
| | probable ALS (N = 37): | | Healthy control | |
| Characteristic | CSF: n = 36, plasma: n = 35 | | subjects: n = 17 | |
| Gender: female/male (ratio) | 9/28 | (0.32) | 8/9 | (0.89) |
| Age at biospecimen sampling: | 65 | (41-81) | 64 | (55-70) |
| median (range) | | | | |
| Symptom duration in months at sampling: | 16.4 | (2.1-55.3) | | |
| median (range) | | | | |
| Inheritance: familial/sporadic (ratio) | 2/35 | (0.057) | | |
| Clinical presentation (revised El Escorial | | | | |
| criteria): | | | | |
| Definite | 23 | | | |
| Probable (including laboratory- | 14 | | | |
| supported probable ALS) | | | | |
| ALSFRS-R score | | | | |

| | |
|---|---|
| Score 40-48, mild to minimal | 22 |
| Score 30-39, moderate to mild | 13 |
| Score 21-29, severe to moderate | 2 |

Average biomarker levels detected in each subgroup are shown in Table 6. In the Table, p-values represent level of significance over corresponding healthy control levels (as determined by Kruskal-Wallis test).

TABLE 6
Biomarker levels

| | | | | | Probable | Definite |
|---|---|---|---|---|---|---|
| | Healthy | Probable | Definite | Healthy | ALS | ALS |
| | Control | ALS CSF | ALS CSF | Control | Plasma | Plasma |
| | CSF | ng/ml | ng/ml | Plasma | ng/ml | ng/ml |
| Biomarkers | ng/ml | [p-value] | [p-value] | ng/ml | [p-value] | [p-value] |
| C5a | 0.05 | 0.30 | 0.35 | 7.70 | 6.71 | 10.10 |
| | | [0.0006] | [<0.0001] | | [n.s.] | [n.s.] |
| sC5b-9 | 5.31 | 13.00 | 14.18 | 176.00 | 161.10 | 287.90 |
| | | [0.0067] | [0.0007] | | [n.s.] | [0.0013] |
| C3a | 2.42 | 4.17 | 3.41 | 64.32 | 64.33 | 108.40 |
| | | [0.0212] | [n.s.] | | [n.s.] | [0.0004] |
| C4a | 30.42 | 41.00 | 26.83 | 387.50 | 648.40 | 1124.00 |
| | | [n.s.] | [n.s.] | | [n.s.] | [<0.0001] |
| Ba | 8.25 | 11.68 | 9.70 | 489.00 | 446.20 | 537.00 |
| | | [ns.] | [n.s.] | | [n.s.] | [n.s.] |
| Bb | 6.50 | 12.00 | 10.00 | 1145.00 | 817.50 | 1180.00 |
| | | [n.s.] | [n.s.] | | [0.0034] | [n.s.] |
| fH | 1828.00 | 2100.00 | 1875.00 | 309000.00 | 335000.00 | 284500.00 |
| | | [n.s.] | [n.s.] | | [n.s.] | [n.s.] |
| fI | 161.80 | 229.50 | 203.70 | 26170.00 | 20300.00 | 27290.00 |
| | | [0.0029] | [n.s.] | | [n.s.] | [n.s.] |

No significant differences in C5 levels were detected between any healthy control or ALS sample group. In plasma samples analyzed, sC5b9, C3a, and C4a levels were significantly elevated in subjects with definite ALS as compared to healthy controls, but not in subjects with probable ALS. In samples from probable ALS subjects, only Bb levels demonstrated a significant difference from healthy control levels. C5a and sC5b9 levels were significantly elevated in CSF samples from subjects with definite or probable ALS as compared to healthy controls. C3a and fI levels were significantly elevated only with probable ALS as compared to CSF from healthy controls.

As observed previously, C5a and sC5b-9 levels produced in the CSF did not correlate with those observed in plasma, further indicating local CNS complement activity in ALS that is independent from activity in the periphery.

In the expanded sample set, 29% of subjects with definite or probable ALS were determined to have BBB impairment (albumin-quotient value>9.0). Average biomarker levels detected in ALS CSF samples (definite and probable) associated with intact (N=26) or impaired (N=10) BBB are shown in Table 7 along with levels from healthy control CSF samples. In the Table, p-values represent level of significance over corresponding healthy control levels (as determined by Kruskal-Wallis test).

TABLE 7
Biomarker levels

| | | BBB-intact | BBB-impaired |
|---|---|---|---|
| | Healthy | ALS CSF | ALS CSF |
| | Control CSF | ng/ml | ng/ml |
| Biomarkers | ng/ml | [p-value] | [p-value] |
| C5 | 122.50 | 91.00 | 202.50 |
| | | [n.s.] | [n.s.] |
| C5a | 0.05 | 0.20 | 0.70 |
| | | [0.0022] | [<0.0001] |
| sC5b-9 | 5.31 | 12.29 | 42.68 |
| | | [0.0131] | [0.0001] |
| C3a | 2.42 | 2.83 | 4.35 |
| | | [n.s.] | [0.0019] |
| C4a | 30.42 | 28.75 | 28.82 |
| | | [n.s.] | [n.s.] |
| Ba | 8.25 | 8.37 | 16.40 |
| | | [n.s.] | [0.0003] |
| Bb | 6.50 | 7.75 | 16.25 |
| | | [n.s.] | [0.0003] |
| fH | 1828.00 | 1803.00 | 2310.00 |
| | | [n.s.] | [0.0269] |
| fI | 161.80 | 197.30 | 263.20 |
| | | [n.s.] | [0.0001] |

Levels of C5a and sC5b-9 were significantly elevated in ALS CFS associated with both intact and impaired BBB, while C3a, Ba, Bb, fH, and fI were only significantly elevated in ALS CSF associated with impaired BBB.

What is claimed is:

1. A method of treating a subject, wherein the subject has or is suspected of having amyotrophic lateral sclerosis (ALS), the method comprising administering a complement inhibitor to the subject, wherein the complement inhibitor reduces, prevents, stabilizes, or reverses one or more effects of ALS experienced by the subject.

2. The method of claim 1, wherein the complement inhibitor comprises a C5 inhibitor.

3. The method of either of claim 1 or 2, wherein the complement inhibitor comprises zilucoplan.

4. The method of any one of claims 1-3, wherein prior to treatment:
subject ALS disease severity is elevated; and/or
subject ALS disease severity is increasing at a specific rate; and
wherein treatment with the complement inhibitor:
stabilizes or reduces subject ALS disease severity; and/or
reduces the specific rate at which subject ALS disease severity is increasing.

5. The method of claim 4, wherein change in subject ALS disease severity is measured by ALS Functional Rating Scale-Revised (ALSFRS-R).

6. The method of any one of claims 1-5, wherein prior to treatment, subject respiratory function is decreased and/or is declining at a specific rate; and wherein treatment with the complement inhibitor stabilizes or improves subject respiratory function and/or reduces the specific rate at which subject respiratory function is declining.

7. The method of claim 6, wherein change in subject respiratory function is assessed by slow vital capacity (SVC).

8. The method of any one of claims 1-7, wherein prior to treatment, subject muscle strength is decreased and/or is declining at a specific rate; and wherein treatment with the complement inhibitor stabilizes or improves subject muscle strength and/or reduces the specific rate at which subject muscle strength is declining.

9. The method of claim 8, wherein change in subject muscle strength is measured by hand held dynamometry (HHD).

10. The method of any of claims 1-9, wherein the complement inhibitor treatment stabilizes or improves subject speech capability; and/or stabilizes or improves a rate of subject speech capability decline.

11. The method of claim 10, wherein the subject speech capability comprises one or more of articulatory precision, speaking rate, percent of speech signal voiced, phonation length, consonant length, and nasality.

12. The method of any of claims 1-11, wherein complement inhibitor treatment stabilizes or improves subject ALS biomarker profile; and/or stabilizes or improves a rate of subject ALS biomarker profile change.

13. The method of claim 12, wherein the subject ALS biomarker profile comprises a biomarker selected from one or more of neurofilament heavy chain (pNfH), neurofilament light chain (NfL), C5, and a complement activity biomarker.

14. The method of claim 13, wherein the complement activity biomarker is selected from one or more of C5a, C5b-9, soluble C5b-9, C3a, C4a, Ba, Bb, Factor I, and Factor H.

15. The method of claim 14, wherein the complement activity biomarker is detected in subject cerebrospinal fluid (CSF), plasma, and/or serum.

16. The method of any of claims 1-15, wherein the subject has or is suspected of having myasthenia gravis.

17. The method of any one of claims 1-16, wherein the subject is positive for at least one autoantibody.

18. The method of claim 17, wherein the at least one autoantibody comprises one or more of anti-acetylcholine receptor antibody, anti-LDL receptor related protein 4, anti-agrin, and anti-muscle associated receptor tyrosine kinase.

19. The method of any one of claims 1-18, wherein the subject has impaired blood brain barrier function.

20. The method of claim 19, wherein the impaired blood brain barrier function is assessed by determining the subject's albumin quotient.

21. The method of any one of claims 1-20, wherein the subject is determined to have definite ALS or probable ALS based on revised El Escorial criteria.

22. A method of diagnosing ALS in a subject, the method comprising detecting and/or quantifying complement activity associated with the subject or a biological sample from the subject and diagnosing the subject with ALS based on complement activity detected and/or quantified.

23. The method of claim 22, wherein complement activity detection and/or quantification comprises detecting and/or quantifying a complement activity biomarker.

24. The method of claim 23, wherein the complement activity biomarker is selected from one or more of C5a, C5b-9, soluble C5b-9, C3a, C4a, Ba, Bb, Factor I, and Factor H.

25. The method of claim 23 or 24, wherein the complement activity biomarker is detected and/or quantified in subject CSF, plasma, and/or serum.

26. A method of monitoring ALS in a subject, the method comprising:
detecting and/or quantifying complement activity associated with:
the subject at an initial time point or over an initial time period; or
a biological sample obtained from the subject at the initial time point or over the initial time period;
detecting and/or quantifying complement activity associated with:
the subject at a subsequent time point or over a subsequent time period; or
a biological sample obtained from the subject at the subsequent time point or over the subsequent time period; and
monitoring ALS in the subject by assessing any change in complement activity detected and/or quantified between the initial time point or period and the subsequent time point or period.

27. The method of claim 26, wherein complement activity detection and/or quantification comprises detecting and/or quantifying a complement activity biomarker.

28. The method of claim 27, wherein the complement activity biomarker is selected from one or more of C5a, C5b-9, soluble C5b-9, C3a, C4a, Ba, Bb, Factor I, and Factor H.

29. The method of claim 27 or 28, wherein the complement activity biomarker is detected and/or quantified in subject CSF, plasma, and/or serum.

30. The method of any one of claims 26-29, wherein the subject is determined to have definite ALS or probable ALS based on revised El Escorial criteria.

31. A method of stratifying subjects with ALS or suspected of having ALS into two or more groups, the method comprising detecting and/or quantifying complement activity associated with each subject or a biological sample from each subject and assigning each subject to at least one of the two or more groups based on complement activity detected and/or quantified.

32. The method of claim 31, wherein complement activity detection and/or quantification comprises detecting and/or quantifying a complement activity biomarker.

33. The method of claim 32, wherein the complement activity biomarker is selected from one or more of C5a, C5b-9, soluble C5b-9, C3a, C4a, Ba, Bb, Factor I, and Factor H.

34. The method of claim 32 or 33, wherein the complement activity biomarker is detected and/or quantified in subject CSF, plasma, and/or serum.

35. The method of claim 34, wherein subjects are assigned to a treatment group based on CSF, plasma, and/or serum complement activity biomarker levels that are elevated in comparison to CSF, plasma, and/or serum complement activity biomarker levels in subjects without ALS.

36. The method of claim 35, wherein the treatment group is characterized by eligibility for complement inhibitor treatment.

37. The method of claim 36, wherein the complement inhibitor treatment comprises zilucoplan treatment.

38. The method of any one of claims 31-37, wherein the subject is determined to have definite ALS or probable ALS based on revised El Escorial criteria.

39. The method of any one of claims 1-21, wherein the dose and/or regimen of complement inhibitor administered is determined based on the level of a complement activity biomarker detected in CSF, plasma, and/or serum obtained from the subject prior to treatment.

40. The method of claim 39, wherein the complement activity biomarker detected in CSF, plasma, and/or serum obtained from the subject prior to treatment is selected from one or more of C5a, C5b-9, soluble C5b-9, C3a, C4a, Ba, Bb, Factor I, and Factor H.

41. The method of any one of claim 1-21, 39, or 40, wherein the dose and/or regimen of complement inhibitor administered is adjusted after one or more prior complement inhibitor administration based on the level of a complement activity biomarker detected in CSF, plasma, and/or serum obtained from the subject after one or more prior administration.

42. The method of claim 41, wherein the complement activity biomarker detected in CSF, plasma, and/or serum obtained from the subject after one or more of prior administration is selected from one or more of C5a, C5b-9, soluble C5b-9, C3a, C4a, Ba, Bb, Factor I, and Factor H.

43. The method of any of claims 39-42, wherein the complement inhibitor administered is zilucoplan.

44. A method of treating a subject having a complement-related indication, wherein the subject has or is suspected of having an impaired blood brain barrier, the method comprising administering a complement inhibitor to the subject, wherein the complement inhibitor reduces, prevents, stabilizes, or reverses one or more effects of the complement-related indication.

45. The method of claim 44, wherein the complement inhibitor comprises a C5 inhibitor.

46. The method of either of claim 44 or 45, wherein the complement inhibitor comprises zilucoplan.

47. The method of any one of claims 44-46, wherein the complement-related indication comprises a neuromuscular inflammatory indication.

48. The method of claim 47, wherein the neuromuscular inflammatory indication comprises ALS.

49. The method of claim 48, wherein the subject is determined to have definite ALS or probable ALS based on revised El Escorial criteria.

50. The method of any one of claims 44-49, wherein the subject is assessed for blood brain barrier impairment by determining the subject's albumin quotient.

51. A method of selecting a subject for treatment with a complement inhibitor, wherein the subject has or is suspected of having ALS, the method comprising:
assessing the presence or level of at least one biomarker in a subject sample, wherein the at least one biomarker is selected from one or more of a neuroaxonal degeneration biomarker, a neuroinflammation biomarker, and a complement activity biomarker; and
selecting the subject for treatment with the complement inhibitor based on the assessment of the presence or level of the at least one biomarker in the subject sample.

52. The method of claim 51, wherein the subject sample comprises a plasma sample and/or a CSF sample.

53. The method of claim 51 or 52, wherein the neuroaxonal degeneration biomarker is pNfH or NfL.

54. The method of any one of claims 51-53, wherein the neuroinflammation biomarker is monocyte chemoattractant protein-1 (MCP-1), chitotriosidase-1 (CHIT1), or chitinase-3-like protein 1 (YKL-40).

55. The method of any one of claims 51-54, wherein the complement activity biomarker is C5a, C5b-9, sC5b-9, C5b6 complex, C3a, C4a, Ba, Bb, Factor I, or Factor H.

56. The method of any one of claims 51-55, wherein the complement inhibitor comprises zilucoplan.

57. The method of claim 56, wherein levels of one or more of Nfl, pNfH, CHIT1, C5a, and sC5b-9 are elevated in the subject sample relative to levels in a subject sample from a subject without ALS.

58. The method of claim 57, wherein the subject sample and the subject sample from a subject without ALS comprise CSF.

59. The method of claim 58, wherein the subject sample is obtained from a subject with impaired blood brain barrier function.

60. The method of claim 59, wherein one or more of C3a, Bb, and Ba are elevated in the subject sample relative to levels in a subject sample from a subject without ALS.

61. The method of claim 57, wherein the subject sample and the subject sample from a subject without ALS comprise plasma.

62. The method of claim 61, wherein C3a is elevated in the subject sample relative to levels in a subject sample from a subject without ALS.

63. A method of treating a subject with ALS or suspected of having ALS, the method comprising:
selecting the subject for treatment with a complement inhibitor according to the method of any one of claims 51-62; and
administering the complement inhibitor to the subject.

64. The method of claim 63, wherein the complement inhibitor comprises zilucoplan.

65. A composition comprising a complement inhibitor for use in a method of treating a subject having or suspected of having ALS, wherein the composition reduces, prevents, stabilizes, or reverses one or more effects of ALS experienced by the subject.

66. The composition for use according to claim 65, wherein the complement inhibitor comprises a C5 inhibitor.

67. The composition for use according to claim 65 or 66, wherein the complement inhibitor comprises zilucoplan.

68. The composition for use according to any one of claims 65-67, wherein prior to treatment:
subject ALS disease severity is elevated; and/or
subject ALS disease severity is increasing at a specific rate; and
wherein the treatment:
stabilizes or reduces subject ALS disease severity; and/or
reduces the specific rate at which subject ALS disease severity is increasing.

69. The composition for use according to claim 68, wherein change in subject ALS disease severity is measured by ALSFRS-R.

70. The composition for use according to any one of claims 65-69, wherein prior to treatment, subject respiratory function is decreased and/or is declining at a specific rate; and wherein treatment with the complement inhibitor stabilizes or improves subject respiratory function and/or reduces the specific rate at which subject respiratory function is declining.

71. The composition for use according to claim 70, wherein change in subject respiratory function is assessed by SVC.

72. The composition for use according to any one of claims 65-71, wherein prior to treatment, subject muscle strength is decreased and/or is declining at a specific rate; and wherein treatment with the composition stabilizes or improves subject muscle strength and/or reduces the specific rate at which subject muscle strength is declining.

73. The composition for use according to claim 72, wherein change in subject muscle strength is measured by HHD.

74. The composition for use according to any one of claims 65-73, wherein the treatment stabilizes or improves subject speech capability; and/or stabilizes or improves a rate of subject speech capability decline.

75. The composition for use according to claim 74, wherein the subject speech capability comprises one or more of articulatory precision, speaking rate, percent of speech signal voiced, phonation length, consonant length, and nasality.

76. The composition for use according to any one of claims 65-75, wherein the treatment stabilizes or improves subject ALS biomarker profile; and/or stabilizes or improves a rate of subject ALS biomarker profile change.

77. The composition for use according to claim 76, wherein the subject ALS biomarker profile comprises a biomarker selected from one or more of pNfH, NfL, C5, and a complement activity biomarker.

78. The composition for use according to claim 77, wherein the complement activity biomarker is selected from one or more of C5a, C5b-9, soluble C5b-9, C3a, C4a, Ba, Bb, Factor I, and Factor H.

79. The composition for use according to claim 78, wherein the complement activity biomarker is detected in subject CSF, plasma, and/or serum.

80. The composition for use according to any one of claims 65-79, wherein the subject has or is suspected of having myasthenia gravis.

81. The composition for use according to any one of claims 65-80, wherein the subject is positive for at least one autoantibody.

82. The composition for use according to claim 81, wherein the at least one autoantibody comprises one or more of anti-acetylcholine receptor antibody, anti-LDL receptor related protein 4, anti-agrin, and anti-muscle associated receptor tyrosine kinase.

83. The composition for use according to any one of claims 65-82, wherein the subject has impaired blood brain barrier function.

84. The composition for use according to claim 83, wherein the impaired blood brain barrier function is assessed by determining the subject's albumin quotient.

85. The composition for use according to any one of claims 65-84, wherein the subject is determined to have definite ALS or probable ALS based on revised El Escorial criteria.

86. A method of diagnosing ALS in a subject, the method comprising detecting and/or quantifying complement activity associated with a biological sample obtained from the subject and diagnosing the subject with ALS based on complement activity detected and/or quantified.

87. The method of claim 86, wherein the step of detecting and/or quantifying complement activity associated with the biological sample comprises detecting and/or quantifying a complement activity biomarker in the biological sample.

88. The method of claim 87, wherein the complement activity biomarker is selected from one or more of C5a, C5b-9, soluble C5b-9, C3a, C4a, Ba, Bb, Factor I, and Factor H.

89. The method of claim 87 or 88, wherein the biological sample comprises subject CSF, plasma, and/or serum.

90. A method of monitoring ALS in a subject, the method comprising:
detecting and/or quantifying complement activity associated with a biological sample obtained from the subject at an initial time point or over an initial time period;
detecting and/or quantifying complement activity associated with a biological sample obtained from the subject at a subsequent time point or over a subsequent time period; and
monitoring ALS in the subject by assessing any change in complement activity detected and/or quantified between:
the biological sample obtained from the subject at the initial time point or over the initial time period; and
the biological sample obtained from the subject at the subsequent time point or over the subsequent time period.

91. The method of claim 90, wherein complement activity detection and/or quantification comprises detecting and/or quantifying a complement activity biomarker.

92. The method of claim 91, wherein the complement activity biomarker is selected from one or more of C5a, C5b-9, soluble C5b-9, C3a, C4a, Ba, Bb, Factor I, and Factor H.

93. The method of any one of claims 90-92, wherein the biological sample obtained from the subject at the initial time point or over the initial time period and/or the biological sample obtained from the subject at the subsequent time point or over the subsequent time period comprises CSF, plasma, and/or serum.

94. The method of any one of claims 90-93, wherein the subject has definite ALS or probable ALS, as assessed based on revised El Escorial criteria.

95. A method of stratifying subjects with ALS or suspected of having ALS into two or more groups, the method comprising detecting and/or quantifying complement activity associated with a biological sample obtained from each subject and assigning each subject to at least one of the two or more groups based on complement activity detected and/or quantified.

96. The method of claim 95, wherein complement activity detection and/or quantification comprises detecting and/or quantifying a complement activity biomarker.

97. The method of claim 96, wherein the complement activity biomarker is selected from one or more of C5a, C5b-9, soluble C5b-9, C3a, C4a, Ba, Bb, Factor I, and Factor H.

98. The method of claim 96 or 97, wherein the biological sample obtained from each subject comprises CSF, plasma, and/or serum.

99. The method of claim 98, wherein the two or more groups comprise a treatment group, wherein the subjects are assigned to the treatment group based on CSF, plasma, and/or serum complement activity biomarker levels that are elevated in comparison to CSF, plasma, and/or serum complement activity biomarker levels in subjects without ALS.

100. The method of claim 99, wherein the treatment group is characterized by eligibility for complement inhibitor treatment.

101. The method of claim 100, wherein the complement inhibitor treatment comprises zilucoplan treatment.

102. The method of any one of claims 95-101, wherein the subject is determined to have definite ALS or probable ALS based on revised El Escorial criteria.

103. The composition for use according to any one of claims 65-85, wherein the dose and/or regimen of the composition is determined based on the level of a complement activity biomarker detected in a CSF sample, plasma sample, and/or serum sample obtained from the subject prior to treatment.

104. The composition for use according to claim 103, wherein the complement activity biomarker detected in the CSF sample, plasma sample, and/or serum sample obtained from the subject prior to treatment is selected from one or more of C5a, C5b-9, soluble C5b-9, C3a, C4a, Ba, Bb, Factor I, and Factor H.

105. The composition for use according to any one of claim 65-85, 103, or 104, wherein the dose and/or regimen of the composition is adjusted after one or more prior composition administration based on the level of a complement activity biomarker detected in a CSF sample, plasma sample, and/or serum sample obtained from the subject after one or more prior composition administration.

106. The composition for use according to claim 105, wherein the complement activity biomarker detected in the CSF sample, plasma sample, and/or serum sample obtained from the subject after one or more prior composition administration is selected from one or more of C5a, C5b-9, soluble C5b-9, C3a, C4a, Ba, Bb, Factor I, and Factor H.

107. The composition for use according to any one of claims 103-106, wherein the composition comprises zilucoplan.

108. A composition comprising a complement inhibitor for use in a method of treating a subject having a complement-related indication, wherein the subject has or is suspected of having an impaired blood brain barrier, wherein the composition reduces, prevents, stabilizes, or reverses one or more effects of the complement-related indication.

109. The composition for use according to claim 108, wherein the complement inhibitor comprises a C5 inhibitor.

110. The composition for use according to claim 108 or 109, wherein the complement inhibitor comprises zilucoplan.

111. The composition for use according to any one of claims 108-110, wherein the complement-related indication comprises a neuromuscular inflammatory indication.

112. The composition for use according to claim 111, wherein the neuromuscular inflammatory indication comprises ALS.

113. The composition for use according to claim 112, wherein the subject is determined to have definite ALS or probable ALS based on revised El Escorial criteria.

114. The composition for use according to any one of claims 108-113, wherein the subject is assessed for blood brain barrier impairment by determining the subject's albumin quotient.

115. A composition comprising a complement inhibitor for use in a method of treating a subject with ALS or suspected of having ALS, wherein the method comprises:
selecting the subject for treatment with the composition according to the method of any one of claims 51-62; and
administering the composition to the subject.

116. The composition for use according to claim 115, wherein the complement inhibitor comprises a C5 inhibitor.

117. The composition for use according to claim 116, wherein the C5 inhibitor comprises zilucoplan.


SEQUENCE LISTING (1 sequence)

SEQ ID NO: 1 - protein, 15 aa, Artificial Sequence
  source: /note=“Description of Artificial Sequence Synthetic peptide”
  source: /note=“N-term Ac”
  SITE (1)..(6): /note=“Lactam bridge between residues”
  MOD_RES (7)..(7): N-methyl-aspartic acid
  MOD_RES (8)..(8): Tert-butylglycine
  MOD_RES (10)..(10): 7-azatryptophan
  MOD_RES (14)..(14): Cyclohexylglycine
  MOD_RES (15)..(15): N-epsilon-(PEG24-gamma-glutamic acid-N-alpha- hexadecanoyl)Lys
KVERFDXXYXEYPXK